SEQUENCE LISTING (30 sequences)

SEQ ID NO: 1 - RNA, 21 nt, Mus musculus
cagaauacuucauaaggaauu

SEQ ID NO: 2 - RNA, 21 nt, Mus musculus
uuccuuaugaaguauucuguu

SEQ ID NO: 3 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 4 - protein, 587 aa, Homo sapiens
MPLDKAMPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 5 - DNA, 5881 bp, Homo sapiens
acactcatgctgcagccttgagccgtccctcgtcctcctctcaggctccctcttgtccacggcgggcgggcgccgagctgctggctatgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggcctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgggctggccattgccgccattgtaattggcattgtcgccctggcctgctccctggctgcctgcgtcggctgttgctgctgcaagaagaggagccaagctgtcctgatgcagatgaaggcacccaatgagtgttaaagaggcaggctggagcagggctggggaatgatgggactggaggacctgggaatttcatctttctgcctccacccctgggtccatggagctttcccgtgattgctctttctggccccagagaaaggtgagcctacctcttcctgacttgcctgattctcccgtagagaagcaggtcgtgccggaccttcctacaatcaggaagatagatccaactggccatggcaaaagccctggggatttccgattcatacccctgggcttccttcgagagggctcttcctccaaatcctccccacctgtcctccaagaacagccttccctgcgcccaggccccctccgggcctctgtagactcagttagtccacagcctgctcacttcgtgggaatagttctccgctgagatagcccctctcgcctaagtattatgtaagttgatttcccttcttttgtttctcttgtttgtgctacggcttgacccagcatgtcccctcaaatgaaagttctccccttgattttctgctcctgaaggcagggtgagttctctcctcaaagaagacttcaaaccatttaactggtttcttaagagccgtcaatcagcctggttttggggatgctatgaaagagagaaggaaaatcatgccgctcagttcctggagacagaagagccgtcatcagtgtctcacttgtgatttttatctggaaaaggaagaaacaccccagcacagcaagctcagccttttagagaaggatatttccaaactgcaaactttgctttgaaaagtttagccctttaaggaatgaaatcatgtagaattttggacttctaaaaacattaaaatcagcttattaatacgggatagagaaagaaatctggtgcctgggggtccctgtgttcacccctagagtttgttttaaaatttttaattgaagcatgtgaagtgtacgtgcagaaaagtgggaacatgatagtgtatggcttggtggattttcacaaactgaacatacctgtgtaatcagcatctagacccagacccagagcatcacaaatatcccccatcctgggcttttcccagaggagatgggggcttctgaagatggacttacctgggacctgccccccatgagccaggacggtccccccacagtcagcctgtgcaaaggccccgtggccaggggtggaggagaatatgtgggtgtggacaggatgggagactgtggcctgaacaggagattttattatatctggagaccctgagagaccctgagacctggggcaccatggctggccaggtcagaagcatcctgactgcagaggtccgtgcagccacaccctcttccctgccagcaagctgtctgcggctcatcggaggcccctccgcctggagccttctatggacgtgatatgcctgtatctgtttttaattttcattcttcacttaggggaagtgaaatcgctcagagatgagatcctttaattgaaaacgaagtgtaacggaatctagtgtctttctaatgtggtaaaattctccatcaacatcacagtcagctggcagctgaacttcagaatctcacttacagcaggcgacacgggggtacaccgatgggtcacactgggtctgggggctccctggagctcctcctgcgtgtggtctggttaggagttgagttgtttgctccagggttattctcctcctcgagtcacagtcacacgaatacctgccttctctggctttcctgctatacacatattcacatggcgctcaagaagttaggctcatggcaacgtgtgtctttctctggacaactggcccagtttacagtgaaatggagaatttcaggtctccacgtctgcccaggaaagaacttcagctgactccacggggatctggaaatccacgaccaatcccgatcggctcttattagctccccgctccacaagacacctgtgctttggaaatccaccaccaatcccgatcggctcttattagctccccgctccacaagacacctgtgatctggaaatctaccaccaatcccgatcggctcttattagctccccgctccacaagacacctgtgacatcctccagggccacaggagcacgtgctgaccagttttcccttccagttcctgcacaaaaagtgtccagagggctgtttgcaaacactagtgcactttgtagcttttcaccctctgtcccagggaatctaggagagatgaggcccgtcagagtcaagagatgtcatccccccagggtctccaaggcatttccacactattggtggcacctggaggacatgcaccaaggcttgccagagccaacaggaagtgagcccagagcatggcacatgagcatcacccgctgatggtggcctgctgtgcctggtgccaacaggggcatcccggcccgtacccctccagacaggaagcatgggtttgcccacagacctgtcgggtgctcctgtgagtggcctccagatgtctttgtgcataggcacaagtgggccagggctggagggaggtgggaaacctcatcatccggtgggccctgccaatcttaacccagaacccttaggtattcctggcagtagccatgacattggagcaccttcctctccagccagaggctgacctgagggccactgtcctcagatgacaccacccaggagcaccctaggtgaggggtgagggcccccttatgtgaacctcttgcctcttcctttctcccatcagagtggttggatggagccattggcctccttttcttcagcgggcccttcaacctctctgcaccatgttgtctggctgaggagctactagaaaagctgagtggagtctcctttccaacaggatgatgcatttgctcaattctcagggctggaatgagccggctggtcccccagaaagctggagtggggtacagagttcagttttcctctctgtttacagctccttgacagtcccacgcccatctggagtgggagctgggagtcagtgttggagaagaaacaacaaaagccaattagaaccactatttttaaaaagtgcttactgtgcacagatactcttcaagcactggacgtggattctctctctagccctcagcacccctgcggtaggagtgccgcctctacccacttgtgatggggtacagaggcacttgctcttctgcatggtgttcaataggctgggagttttatttatctcttcaaactttgtacaagagctcatggcttgtcttgggctttcgtcattaaaccaaaggaaatggaagccattcccctgttgctctccttagtcttggtcatcagaacctcacttggtaccatatagatcaaaagctttgtaaccacaggaaaaaataaactcttccatcccttaaagaatagaatagtttgtccctctcatgggaattgggctgtatgtatattgttcttcctccttagaatttagagatacaagagttctacttagaacttttcatggacacaatttccacaacctttcagatgctgatgtagagctattgggaaagaacttccaaactcaggaagtttgcagagagcagacagctagagataactcgggacccagagttggtcgacagatgttagatgtatcctagcttttagctataaaccactcaaagattcagcccccagatcccacagtcagaactgaatctgcgttgttgggaagccagcagtggccttgggaaggaagccatggctgtggttcagagagggtgggctggcaagccacttccggggaaaactccttccgccccaggtttcttcttctcttaaggagagattgttctcaccaacccgctgccttcatgctgccttcaaagctagatcatgtttgccttgcttagagaattactgcaaatcagccccagtgcttggcgatgcatttacagatttctaggccctcagggttttgtagagtgtgagccctggtgggcagggttggggggtctgtcttctgctggatgctgcttgtaatccatttggtgtacagaatcaacaataaataatatacatgta

SEQ ID NO: 6 - DNA, 5938 bp, Homo sapiens
acactcatgctgcagccttgagccgtccctcgtcctcctctcaggctccctcttgtccacggcgggcgggcgccgagctgctgatgtgtccctagcataacctggccccattcctttgcatctagatgcctttggacaaggctatgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggcctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgggctggccattgccgccattgtaattggcattgtcgccctggcctgctccctggctgcctgcgtcggctgttgctgctgcaagaagaggagccaagctgtcctgatgcagatgaaggcacccaatgagtgttaaagaggcaggctggagcagggctggggaatgatgggactggaggacctgggaatttcatctttctgcctccacccctgggtccatggagctttcccgtgattgctctttctggccccagagaaaggtgagcctacctcttcctgacttgcctgattctcccgtagagaagcaggtcgtgccggaccttcctacaatcaggaagatagatccaactggccatggcaaaagccctggggatttccgattcatacccctgggcttccttcgagagggctcttcctccaaatcctccccacctgtcctccaagaacagccttccctgcgcccaggccccctccgggcctctgtagactcagttagtccacagcctgctcacttcgtgggaatagttctccgctgagatagcccctctcgcctaagtattatgtaagttgatttcccttcttttgtttctcttgtttgtgctacggcttgacccagcatgtcccctcaaatgaaagttctccccttgattttctgctcctgaaggcagggtgagttctctcctcaaagaagacttcaaaccatttaactggtttcttaagagccgtcaatcagcctggttttggggatgctatgaaagagagaaggaaaatcatgccgctcagttcctggagacagaagagccgtcatcagtgtctcacttgtgatttttatctggaaaaggaagaaacaccccagcacagcaagctcagccttttagagaaggatatttccaaactgcaaactttgctttgaaaagtttagccctttaaggaatgaaatcatgtagaattttggacttctaaaaacattaaaatcagcttattaatacgggatagagaaagaaatctggtgcctgggggtccctgtgttcacccctagagtttgttttaaaatttttaattgaagcatgtgaagtgtacgtgcagaaaagtgggaacatgatagtgtatggcttggtggattttcacaaactgaacatacctgtgtaatcagcatctagacccagacccagagcatcacaaatatcccccatcctgggcttttcccagaggagatgggggcttctgaagatggacttacctgggacctgccccccatgagccaggacggtccccccacagtcagcctgtgcaaaggccccgtggccaggggtggaggagaatatgtgggtgtggacaggatgggagactgtggcctgaacaggagattttattatatctggagaccctgagagaccctgagacctggggcaccatggctggccaggtcagaagcatcctgactgcagaggtccgtgcagccacaccctcttccctgccagcaagctgtctgcggctcatcggaggcccctccgcctggagccttctatggacgtgatatgcctgtatctgtttttaattttcattcttcacttaggggaagtgaaatcgctcagagatgagatcctttaattgaaaacgaagtgtaacggaatctagtgtctttctaatgtggtaaaattctccatcaacatcacagtcagctggcagctgaacttcagaatctcacttacagcaggcgacacgggggtacaccgatgggtcacactgggtctgggggctccctggagctcctcctgcgtgtggtctggttaggagttgagttgtttgctccagggttattctcctcctcgagtcacagtcacacgaatacctgccttctctggctttcctgctatacacatattcacatggcgctcaagaagttaggctcatggcaacgtgtgtctttctctggacaactggcccagtttacagtgaaatggagaatttcaggtctccacgtctgcccaggaaagaacttcagctgactccacggggatctggaaatccacgaccaatcccgatcggctcttattagctccccgctccacaagacacctgtgctttggaaatccaccaccaatcccgatcggctcttattagctccccgctccacaagacacctgtgatctggaaatctaccaccaatcccgatcggctcttattagctccccgctccacaagacacctgtgacatcctccagggccacaggagcacgtgctgaccagttttcccttccagttcctgcacaaaaagtgtccagagggctgtttgcaaacactagtgcactttgtagcttttcaccctctgtcccagggaatctaggagagatgaggcccgtcagagtcaagagatgtcatccccccagggtctccaaggcatttccacactattggtggcacctggaggacatgcaccaaggcttgccagagccaacaggaagtgagcccagagcatggcacatgagcatcacccgctgatggtggcctgctgtgcctggtgccaacaggggcatcccggcccgtacccctccagacaggaagcatgggtttgcccacagacctgtcgggtgctcctgtgagtggcctccagatgtctttgtgcataggcacaagtgggccagggctggagggaggtgggaaacctcatcatccggtgggccctgccaatcttaacccagaacccttaggtattcctggcagtagccatgacattggagcaccttcctctccagccagaggctgacctgagggccactgtcctcagatgacaccacccaggagcaccctaggtgaggggtgagggcccccttatgtgaacctcttgcctcttcctttctcccatcagagtggttggatggagccattggcctccttttcttcagcgggcccttcaacctctctgcaccatgttgtctggctgaggagctactagaaaagctgagtggagtctcctttccaacaggatgatgcatttgctcaattctcagggctggaatgagccggctggtcccccagaaagctggagtggggtacagagttcagttttcctctctgtttacagctccttgacagtcccacgcccatctggagtgggagctgggagtcagtgttggagaagaaacaacaaaagccaattagaaccactatttttaaaaagtgcttactgtgcacagatactcttcaagcactggacgtggattctctctctagccctcagcacccctgcggtaggagtgccgcctctacccacttgtgatggggtacagaggcacttgctcttctgcatggtgttcaataggctgggagttttatttatctcttcaaactttgtacaagagctcatggcttgtcttgggctttcgtcattaaaccaaaggaaatggaagccattcccctgttgctctccttagtcttggtcatcagaacctcacttggtaccatatagatcaaaagctttgtaaccacaggaaaaaataaactcttccatcccttaaagaatagaatagtttgtccctctcatgggaattgggctgtatgtatattgttcttcctccttagaatttagagatacaagagttctacttagaacttttcatggacacaatttccacaacctttcagatgctgatgtagagctattgggaaagaacttccaaactcaggaagtttgcagagagcagacagctagagataactcgggacccagagttggtcgacagatgttagatgtatcctagcttttagctataaaccactcaaagattcagcccccagatcccacagtcagaactgaatctgcgttgttgggaagccagcagtggccttgggaaggaagccatggctgtggttcagagagggtgggctggcaagccacttccggggaaaactccttccgccccaggtttcttcttctcttaaggagagattgttctcaccaacccgctgccttcatgctgccttcaaagctagatcatgtttgccttgcttagagaattactgcaaatcagccccagtgcttggcgatgcatttacagatttctaggccctcagggttttgtagagtgtgagccctggtgggcagggttggggggtctgtcttctgctggatgctgcttgtaatccatttggtgtacagaatcaacaataaataatatacatgta

SEQ ID NO: 7 - protein, 449 aa, Artificial Sequence
  Synthetic polypeptide
EVQLVQSGAEVKKPGASVKVSCKASGYKFSSYWIEWVKQAPGQGLEWIGILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNVYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Synthetic polypeptide
DIQMTQSPSSLSASVGDRVTITCRASQDISNYLNWYQQKPGGAVKFLIYITSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 120 aa, Artificial Sequence
  recombinant antibody sequence
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 10 - protein, 107 aa, Artificial Sequence
  recombinant antibody
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 11 - protein, 107 aa, Artificial Sequence; identical to SEQ ID NO: 10
  Synthetic polypeptide

SEQ ID NO: 12 - protein, 110 aa, Artificial Sequence
  Synthetic polypeptide
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 13 - protein, 121 aa, Artificial Sequence
  Synthetic polypeptide
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 14 - protein, 107 aa, Artificial Sequence
  synthetic polypeptide
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 15 - protein, 119 aa, Artificial Sequence
  synthetic polypeptide
EVQLVQSGAEVKKPGSSVKVSCKASGFTFTDYYIHWVKQAPGQGLEWIGLVYPYIGGSSYNQQFKGKATLTVDTSTSTAYMELSSLRSEDTAVYYCARGDNNYDAMDYWGQGTTVTVSS

SEQ ID NO: 16 - protein, 110 aa, Artificial Sequence
  synthetic polypeptide
DIVLTQSPDSLAVSLGERATISCRASQSVSTSTYNYMHWYQQKPGQPPKLLVKYASNLESGVPDRFSGSGSGTDFTLTISSLQAEDVAVYYCHHTWEIRTFGGGTKVEIK

SEQ ID NO: 17 - protein, 120 aa, Artificial Sequence
  synthetic polypeptide
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 18 - protein, 112 aa, Artificial Sequence
  synthetic polypeptide
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 19 - protein, 120 aa, Artificial Sequence
  synthetic polypeptide
QVQLQQSGAELVRPGTSVKISCKASGYDFTNYWLGWVKQRPGHGLEWIGDIYPGGGNTYYNEKLKGKATLTADKSSSTAYIHLISLTSEDSSVYFCARWGDKKGNYFAYWGQGTLVTVSA

SEQ ID NO: 20 - protein, 107 aa, Artificial Sequence
  synthetic polypeptide
QIVLTQSPAIMSASLGERVTMTCTASSSVYSSYLHWYQQKPGSSPKLWIYSTSNLASGVPGRFSGSGSGTSYSLTISSMEAEDAATYYCHQYHRSPTFGGGTKLEIK

SEQ ID NO: 21 - protein, 122 aa, Artificial Sequence
  synthetic polypeptide
QIQLVQSGPELKKPGETVKISCKASGFAITNFGMNWVKQAPGKGLKWMGWINLYTGEPTFADDFKGRFAFSLETSASTAYLQINNLKNEDTVIYFCARKGETYYRYDGFAYWGQGTLVTVSA

SEQ ID NO: 22 - protein, 112 aa, Artificial Sequence
  synthetic polypeptide
DIVMTQAAPSVPVTPGESVSISCRSSKSLLHSNGNTHLYWFLQRPGQSPQLLIYRMSNLASGVPDRFSGSGSGTAFTLRISRVEAEDVGVYYCMQLLEYPYTFGGGTKLEIE

SEQ ID NO: 23 - protein, 450 aa, Artificial Sequence
  synthetic polypeptide
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 449 aa, Artificial Sequence
  synthetic polypeptide
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 449 aa, Artificial Sequence
  synthetic polypeptide
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 449 aa, Artificial Sequence
  synthetic polypeptide
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 448 aa, Artificial Sequence
  synthetic polypeptide
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 448 aa, Artificial Sequence
  synthetic polypeptide
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 217 aa, Artificial Sequence
  synthetic polypeptide
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 449 aa, Artificial Sequence
  synthetic polypeptide
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0212282 A1
(43) Pub. Date: Jul. 6, 2023

(54) METHODS AND COMPOSITIONS FOR TREATING, PREVENTING THE ONSET AND/OR SLOWING PROGRESSION OF OSTEOARTHRITIS

(71) Applicant: New York Society for the Relief of the Ruptured and Crippled, maintaining the Hospital for Special, New York, NY (US)

(72) Inventors: Miguel Otero Adrán, New York, NY (US); Purva Singh, Plainsboro, NJ (US)

(21) Appl. No.: 18/000,010

(22) PCT Filed: May 28, 2021
(86) PCT No.: PCT/US2021/034734

§ 371 Date: Nov. 28, 2022

Related U.S. Application Data
(60) Provisional application No. 63/032,013, filed on May 29, 2020.

(51) Int. Cl. C07K 16/28; A61K 9/00; A61K 9/14; A61P 19/02

(52) U.S. Cl. CPC C07K 16/28; A61K 9/0019; A61K 9/141; A61P 19/02; A61K 2039/545

(57) ABSTRACT

Methods and compositions for treating, inhibiting, and/or preventing the progression of osteoarthritis comprise compositions that blocks or inhibits the expression, induction, activity, or signaling of LRRC15 protein or the expression, transcription or activity of the LRRC15 gene and administering such compositions to a human subject having osteoarthritis and in need thereof.

### INCORPORATION-BY REFERENCE OF MATERIAL SUBMITTED IN ELECTRONIC FORM

Applicant hereby incorporates by reference the Sequence Listing material filed in electronic form herewith. This file is labeled H552019-025PCT_ST25.txt”, was created on May 27, 2021, and is 76 KB in size. It is incorporated by reference herein. Table 1 below lists the SEQ ID Nos and the type of sequence it references.

### STATEMENT REGARDING FEDERALLY SPONSORED RESEARCH OR DEVELOPMENT

This invention was made with government support under grant number R21 AG049980-01A1 awarded by the National Institutes of Health. The government has certain rights in this invention.

### BACKGROUND OF THE INVENTION

Osteoarthritis (OA) is a major cause of pain and disability worldwide and represents a burden on health from both morbidity and cost. OA is characterized by irreversible structural and functional changes in articular cartilage associated with phenotypic instability of articular chondrocytes. Cartilage degradation is a hallmark of OA disease, but the mechanisms initiating cartilage destruction are still not clearly identified and no successful therapeutic intervention exists. This is in part because of the difficulty of identifying early-stage disease, and of retrieving mechanistic information from early-stage human clinical material. Use of human late-stage specimens impedes analyses of early disease stages and a detailed understanding of the mechanism driving disease initiation and progression. Consequently, the use of adequate models that mimic aspects of the human disease is essential to understand the disease and for the development of successful therapeutic approaches.

The mechanisms involved in arthritic joint pain are complicated, while structural pathologies, neuronal mechanisms of pain, and general factors such as obesity and genetic factors shall all take part in the consequence of joint pain. Central and peripheral sensitizations of the nociceptive system are extensively proposed mechanisms of neuronal causes of OA joint pain. The complex pathogenesis of OA has resulted in significant challenges for the development of therapeutic strategies, in part because studies with late-stage human specimens do not provide information about early disease mechanisms. The characteristic change of OA is cartilage breakdown, but a growing consensus has proposed OA as a disease of the whole joint, involving all joint tissues.

Chondrocytes are the unique cell type residing in articular cartilage and are responsible for maintaining its structural and functional integrity. During OA, chondrocytes undergo abnormal activation and severe phenotypic modulation, displaying dysregulated expression and activities of matrix-degrading enzymes and abnormal production of matrix structural proteins, along with features that resemble hypertrophy- and fibroblast-like phenotypes. As part of these phenotypic alterations, recent studies focused on DNA methylation patterns have reported epigenomic changes in OA cartilage, including age- and disease-related epigenetic features, and distinct clusters of OA patients.

DNA methylation is one of the principal mechanisms by which cells maintain stable phenotypes and stable chromatin configurations. Altered DNA methylation is associated with abnormal gene expression in different pathologies, including human OA. Changes in DNA methylation (epigenetic changes) are present in late-stage human OA cartilage. US Patent Publication No. US2013/0129668 (Firestein) discussed a method for diagnosing arthritis, including OA, by determining whether at least 2 nucleic acid loci or at least 2 genes in a sample from the subject have methylation states indicative of OA. However, the two loci were selected from hundreds of genes listed in this disclosure, which provided little direction.

Currently, there are no efficacious non-surgical alternatives to joint replacement, e.g., total knee replacement for patients with OA. Therapies currently simply address pain and inflammation with anti-inflammatory treatments, which are known to have some side effects and are not successful at retarding the progression of OA.

A continuing need in the art exists for new and effective tools and methods for targeting the early phases of the disease and thereby avoiding the irreversible cartilage destruction observed in late-stage disease. Additionally, minimally invasive therapies are needed for treatment of OA.

### SUMMARY OF THE INVENTION

Therapies that specifically modulate LRRC15 gene expression or LRRC15 protein level and activity are provided herein as minimally invasive and early phases disease-targeting for OA in response to the outstanding need in the art.

In one aspect, a method of treating or reducing the progression of OA comprises administering to a subject having OA an effective amount of a composition that blocks, antagonizes or inhibits the expression, induction, activity, or methylation of the LRRC15 gene or binds, blocks, antagonizes or inhibits the activity or signaling of the LRRC15 protein in vivo.

In another aspect, a method of treating an arthritic joint comprising injecting into the joint of a mammalian subject having osteoarthritis an effective amount of a composition that blocks, antagonizes or inhibits the expression, induction, activity, methylation, of the LRRC15 gene or binds, blocks, antagonizes or inhibits the activity or signaling of LRRC15 protein in vivo. In one embodiment, this method involves local administration of the compositions.

In another aspect, a composition for use in treating or reducing the progression of OA comprises an effective amount of a composition that blocks, antagonizes or inhibits the expression, induction, activity, or methylation, of the LRRC15 gene or binds, blocks, antagonizes or inhibits the activity or signaling of LRRC15 protein in vivo. In one embodiment, this composition comprises an LRRC15 inhibitor associated with a suitable nanocarrier. In certain embodiments, this composition is formulated for local, rather than systemic, administration.

In still another embodiment, the invention provides a method for detecting early stages of OA comprising a step of identifying the presence or level of LRRC15 protein in biological samples from a subject. This method permits intervention of OA at an early stage.

In yet another aspect, the present invention provides compositions and methods for treating OA at an early stage as described further in the following detailed description and preferred embodiments thereof.

### BRIEF DESCRIPTION OF THE DRAWINGS

FIG. 1 is an experimental outline of the surgical induction of OA using the destabilization of the medial meniscus model (DMM) and downstream analyses performed at 4 and 12 weeks after surgery (histology, immunohistochemistry, and RNA and DNA isolation for RNAseq and RRoxBS, respectively).

FIG. 2 is a schematic of Reduced Representation of Oxidative Bisulfite Sequencing. It is a well-known two-step process. Bisulfite treatment converts unmethylated cytosine (C) to uracil (U), whereas methylated cytosines (5 mC and 5 hmC) remain unchanged. Unmethylated cytosines are recognized as thymines during sequencing. To separate cytosine methylation (5 mC) from hydroxymethylation (5 hmC), an oxidation step is added that converts 5 hmC to formylcytosine (5 fC), which is converted to uracil by the bisulfite treatment, and recognized as thymine after sequencing. Comparison of the DNA before and after oxidation allows the recognition and separation of methyl and hydroxymethyl cytosines.

FIGS. 3A to 3F show data from RNA-seq analyses in mouse cartilage isolated after surgical induction of OA. FIGS. 3A and 3B are representative Safranin 0-stained histological sections of mouse cartilage at 4 weeks (FIG. 3A; n=9/ea) and at 12 weeks (FIG. 3B; n=8/ea) weeks after surgical induction of OA. FIGS. 3C and 3D are graphs that represent the OARSI (SUM) cartilage degradation scores at 4 weeks (FIG. 3C) and at 12 weeks (FIG. 3D). *p<0.05 and ***p<0.001 by Mann-Whitney. FIG. 3E is a Volcano plot representing significantly differentially expressed genes (red, adjusted p-value <0.05) identified by RNA-seq analyses of microdissected cartilage tissues retrieved at 4 and 12 weeks after DMM surgery (n=3 per condition and per time point). Log fold-changes in the OA (DMM operated) vs. control limbs are shown for each time point. FIG. 3F is a network analyses showing genes with increased (red) and decreased (green) expression in OA cartilage from top enriched functions in cartilage tissues after surgical induction of OA.

FIG. 4 is a schematic showing functions relevant to cartilage development that are enriched in early OA. Gene ontology (GO) enriched functions such as, ossification, muscle hypertrophy, extracellular matrix organization—indicated by color symbols, along with differentially expressed genes belonging to these functional categories.

FIGS. 5A-5E provide data on RRoxBS analyses that identified changes in 5 mC and 5 hmC in mouse cartilage isolated after surgical induction of OA. FIG. 5A shows changes in gene-associated differentially methylated regions (DMRs, 25% difference in methylation and q value <0.05) in microdissected cartilage at 4 and 12 weeks after induction of OA. FIGS. 5B and 5C are overlapping significantly enriched (5B) Biological Processes and (5C) Molecular Functions comparing gene expression (RNA-seq) and DNA methylation (RRoxBS, 5 mC). FIGS. 5D and 5E are representations of the (5D) Biological Processes (top 40) and (5E) Molecular Functions significantly enriched (FDR<0.05) using differentially methylated regions in OA vs. non-OA mouse cartilage samples.

FIGS. 6A-6F provide data showing that the LRRC15 gene is differentially methylated and differentially expressed in mouse OA cartilage. FIG. 6A shows a co-representation of differential expression (y axis, shown as mean Log Fold Change) and differential methylation (x axis, shown as mean differential methylation in gene associated DMRs) of genes with differential expression and methylation. The LRRC15 gene is highlighted in red as the gene with the highest correlation between increased expression and reduced 5 mC. FIGS. 6B and 6C, respectively, are RTqPCR analyses of LRRC15 mRNA (6B) and Lrrc17 mRNA (6C) in mouse cartilage samples at 4 weeks after surgical induction of OA (n=3/ea). Data are shown as fold-change vs. controls (set as 1). *p<0.05 by t-test. FIGS. 6D and 6E are Venn diagrams depicting unique and overlapping differentially expressed genes (DEGs) and differentially methylated regions (DMRs) obtained from our dataset using microdissected cartilage after DMM and published human datasets from human OA cartilage using (6D) structurally intact and eroded cartilage and (6E) healthy and OA cartilage samples. FIG. 6F is a network analysis representing the interaction of LRRC15 with other genes with differential methylation and expression at 4 weeks after surgical induction of OA.

FIGS. 7A-7N show that LRRC15 gene expression is induced by cytokine stimulation and DNA demethylation and contributes to the IL-1β-induced gene expression in mouse chondrocytes in vitro. FIGS. 7A-7C, respectively, are RTqPCR analyses showing (7A) IL-1β-induced LRRC15 expression in human primary chondrocytes (n=4); (7B) IL-1β-induced (n=4) and (7C) TNFα-induced (n=3) LRRC15 expression in mouse primary chondrocytes. FIG. 7D is a Western blotting analysis of the IL-1β-induced LRRC15 protein in mouse primary chondrocytes. FIG. 7E is a quantification of the immunoblot (n=3). FIG. 7F is a RTqPCR analyses of mouse chondrocytes (n=3) treated with 5-Aza-2′-deoxycytidine and trichostatin (labeled as 5-aza) for 72 hours, showing increased LRRC15 expression. Data are shown as fold-change vs. unstimulated controls (set as 1). *p<0.05, **p<0.01 and ***p<0.001 by t-test. FIGS. 7G-7N, respectively, are RTqPCR analyses in cells transfected with non-targeting control siRNA (siControl) or siRNA against LRRC15 (siLRRC15), evaluating (7G) LRRC15 (7H) Col2a1, (7I) Elf3 (7J) Mmp3, (7K) Mmp13, (7L) Mmp10, (7M) Nos2, and (7N) Ptgs2 mRNA in cells left untreated (vehicle, ctrl) or treated with 1 ng/ml of IL-1β for 72 h. *p<0.05, **p<0.01 and ***p<0.001 by ANOVA followed by Tukey's test.

FIGS. 8A-8D show the results from preliminary experiments where long-term cytokine treatment promotes long-term effects in the LRRC15 expression in vitro. FIG. 8A shows a schematic outline of long term treatment with IL1β and DNA demethylation leading to increased LRRC15 expression. (Left) Experimental outline using mouse chondrocytes untreated or treated long-term with IL-1β for 2 weeks, with addition of fresh IL-1β indicated using arrowheads. After 2 weeks of treatment, cells were detached and replated for two additional weeks (indicated with 2w-P). FIG. 8B is a graph underneath the outline represents RTqPCR analyses of the reduced expression of DNA methyl transferases (Dntm) 1, 3a and 3b after 72 h with IL-1β relative to untreated controls (dotted lines). FIGS. 8C and 8D, respectively, show graphs of the results produced when the LRRC15 mRNA expression was evaluated at 72 h after IL-1β treatment (8C), and in cells replated and cultured for additional 2 weeks without IL-1β (2w-P) (8D).

FIGS. 9A-9D show that LRRC15 expression is increased in human and mouse OA infrapatellar fat pads. FIG. 9A shows histological images (H/E-stained) of infrapatellar fat tissues retrieved from non-OA and OA patients showing fibrotic-like changes in OA. FIG. 9B shows a Volcano plot representing differentially expressed genes identified by RNAseq in OA infrapatellar fat pad samples vs. non-OA controls, highlighting the increased expression of LRRC15, TGFb1 and MMP13. FIG. 9C provides histological images of mouse non-OA (ctrl) and OA (load) infrapatellar fat pad tissues. FIG. 9D is a RTqPCR analyses from RNA isolated from mouse non-OA (ctrl) and OA (load) infrapatellar fat pad tissues showing increased LRRC15 mRNA in OA samples.

### DETAILED DESCRIPTION

Methods and compositions for the treatment and retardation of the progression of osteoarthritis as described below are based upon the inventors' theory that progressive time-dependent changes in DNA methylation patterns are driving early phenotypic and functional changes in articular chondrocytes, and therefore are part of the mechanisms that contribute to OA onset and progression. The inventors have identified LRRC15 as a gene with increased expression correlated with hypomethylation in early stages of osteoarthritis (OA). The inventors confirmed that LRRC15 protein is present in human and murine OA cartilage, in agreement with studies showing increased LRRC15 mRNA in human OA cartilage. As shown in the examples below, the inventors' integrative analyses showed that the structural progression of OA is accompanied by transcriptomic and dynamic epigenomic changes in articular cartilage. The inventors found that LRRC15 is differentially methylated and expressed in OA cartilage, and that it contributes to the cytokine-driven responses of OA chondrocytes. Such understanding of the role of LRRC15 in cartilage homeostasis and osteoarthritis supports that LRRC15 is a therapeutic target, such as provided by the methods and compositions described herein.

### Components, Compositions and Definitions

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

As used herein, the terms “Patient” or “subject” or “individual” means a mammalian animal, including a human, a veterinary or farm animal, a domestic animal or pet, and animals normally used for clinical research. In one embodiment, the subject of these methods and compositions is a human. In one embodiment, the subject has OA. In another embodiment, the subject has an early stage of OA and has yet to be treated with any therapy. In another embodiment, the subject has OA and is being treated with conventional methodologies, e.g., administration of anti-inflammatories, but is not responding to the treatment optimally or in a manner sufficient to achieve a sufficient therapeutic benefit. In another embodiment, the subject has advanced OA beyond the early stages.

“LRRC15” (leucine-rich repeat-containing protein 15) is a cell surface protein that has been reported to exist in two isoforms in humans: one containing 587 amino acids (NP_001128529.2 SEQ ID NO: 4) encoded by the gene sequence of 5938 nucleotides (SEQ ID NO: 6; NM_001135057.3) and another containing 581 amino acids (NP_570843.2; SEQ ID NO: 3) encoding by the gene sequence of 5881 nucleotides (SEQ ID NO: 5; NM_130830.5) that is truncated at its N-terminus as compared to the longer isoform. The amino acid sequences and nucleic acid sequences encoding the LRRC15 of both isoforms are publicly available, e.g., see U.S. Pat. No. 10,195,209 and the figures and sequence listing, incorporated by reference herein. Also publicly known are non-human mammalian forms of the LRRC15 gene and LRRC15 protein. For ease of discussion, human LRRC15 is abbreviated herein as “huLRRC15.” This abbreviation is intended to refer to either isoform. U.S. Pat. No. 10,195,209 suggested that antibodies to LRRC15 are useful in the treatment of a solid tumors for certain cancers, such as sarcomas, melanomas and brain cancers (e.g., gliomas, such as glioblastoma).

By the general terms “blocker”, “inhibitor” or “antagonist” is meant agents, compounds, constructs, small molecules, or compositions that inhibit, either partially or fully, the activity, expression, transcription or production of a target molecule, e.g., the protein LRRC15 or the LRRC15 gene as used herein. In certain embodiments, such antagonists are capable of interrupting the expression, transcription, or activity of the LRRC15 gene in vivo or the activity and function of the LRRC15 protein in vivo. In one embodiment, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

The term “salts” when used to describe compositions described herein includes salts of the specific LRRC15 antagonist compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg, Ca) or organic (such as trialkyl ammonium) salts of acidic residues such as carboxylic acids; and the like. The salts of compounds described or referenced herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The “pharmaceutically acceptable salts” of compounds described herein or incorporated by reference include a subset of the “salts” described above which are, conventional non-toxic salts of the parent compound formed, for example, from non-toxic inorganic or organic acids. Lists of suitable salts are found in Remington, J. P., Beringer, P. (2006). Remington: The Science and Practice of Pharmacy. United Kingdom: Lippincott Williams & Wilkins, and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By the term “prodrug” is meant a compound or molecule or agent that, after administration, is metabolized (i.e., converted within the body) into the parent pharmacologically active molecule or compound, e.g., an active LRRC15 inhibitor or antagonists. Prodrugs are substantially, if not completely, in a pharmacologically inactive form that is converted or metabolized to an active form (i.e., drug)—such as within the body or cells, typically by the action of, for example, endogenous enzymes or other chemicals and/or conditions. Instead of administering an active molecule directly, a corresponding prodrug is used to improve how the composition/active molecule is absorbed, distributed, metabolized, and excreted. Prodrugs are often designed to improve bioavailability or how selectively the drug interacts with cells or processes that are not its intended target. This reduces adverse or unintended, undesirable or severe side effects of the active molecule or drug.

By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The antibodies useful in the methods are preferably “immunologically specific”, which refers to proteins/polypeptides, particularly antibodies, that bind to one or more epitopes of a protein or compound of interest, but which do not substantially recognize and bind other molecules in a sample containing a mixed population of antigenic biological molecules.

The antibodies of the instant invention may be further modified. For example, the antibodies may be humanized Methods of humanizing antibodies of non-human origin are well-known in the art. See, for example, without limitation, U.S. Pat. Nos. 7,566,771, 7,262,050, 7,244,832, 7,244,615, 7,022,500, 5,693,762, 6,407,213 and 6,054,297, among many others. In a particular embodiment, the heavy and/or light chain sequences of the antibodies (or only the CDRs thereof) are inserted into a selected backbone or framework of a different antibody or antibody fragment construct. For example, the variable light domain and/or variable heavy domain of the antibodies of the instant invention may be inserted into another antibody construct, e.g., into a different IgG isotype framework or a framework of another selected antibody isotype. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one cell penetrating peptide, detectable agent, imaging agent, or contrast agent. The antibodies useful herein may also comprise at least one purification tag (e.g., a His-tag). In a particular embodiment, the antibody is conjugated to a cell penetrating peptide.

Anti-LRRC15 antibodies are available from a number of commercial sources, including EPR8188(2) (Abcam), N1N3 (GeneTex), ARP50292_P050 (Aviva Systems Biology), antibodies simply designated as LRRC15 Antibody from LifeSpan BioSciences, Inc., Thermo Fisher Scientific, ProSci, Inc., Novus Biologicals, Biorbyt, Cusabio Technology LLC, Bioss Inc, Sigma-Aldrich). Fitgerald Industries International has both an LRRC15 antibody and an LRRC15 blocking peptide. Abbvie further has an LRRC15 antibody-tubulin inhibitor monomethyl auristatin E drug conjugate (ABBV-085) currently in clinical trials for the treatment of osteosarcoma. See P. Hingorani et al, ABBV-085, Antibody-Drug Conjugate Targeting LRRC15, Is Effective in Osteosarcoma: A Report by the Pediatric Preclinical Testing Consortium, Mol Cancer Ther Mar. 1, 2021, 20(3): 535-540. These available antibodies are expected to be useful in the methods described herein.

Certain exemplary LRRC15 antagonists include, without limitation, anti-LRRC15 antibodies and LRRC15 binding fragments thereof, including the antibody drug conjugates defined in U.S. Pat. No. 10,195,209, incorporated by reference. The LRRC15 binding fragments include any moiety capable of specifically binding huLRRC15. LRRC15 antibodies or binding fragments can be used both to target OA chondrocytes and inhibit the protein and also as a conjugate for other antibody that needs to be targeted to OA chondrocytes (antibody-antibody conjugate). Similarly, small peptides/inhibitory small molecules that can be tested for blocking LRRC15 activity based on LRRC15 conformation models and sequence can be used in the methods and compositions described herein.

The anti-LRRC15 antibodies described in U.S. patent Ser. No. 10/195,209 and useful in this method include antibodies having a VH chain comprising the sequence of SEQ ID NO:9 and a VL chain comprising the sequence of SEQ ID NO:10, a VH chain comprising the sequence of SEQ ID NO:11 and a VL chain comprising the sequence of SEQ ID NO:12, a VH chain comprising the sequence of SEQ ID NO:13 and a VL chain comprising the sequence of SEQ ID NO:14, a VH chain comprising the sequence of SEQ ID NO:15 and a VL chain comprising the sequence of SEQ ID NO:16, a VH chain comprising the sequence of SEQ ID NO:17 and a VL chain comprising the sequence of SEQ ID NO:18, a VH chain comprising the sequence of SEQ ID NO:19 and a VL chain comprising the sequence of SEQ ID NO:20, or a VH chain comprising the sequence of SEQ ID NO:21 and a VL chain comprising the sequence of SEQ ID NO:22.

In one embodiment, the antibody or fragment comprises a heavy chain variable sequence of SEQ ID NO: 9, 11, 13, 15, 16, 19 or 21. In another embodiment antibody or fragment comprises a light chain of SEQ ID NO: 10, 12, 14, 16, 18, 20, or 22. In another embodiment, the antibody or fragment comprises a heavy chain amino acid sequence of SEQ ID NOS: 7, 23, 24 or 25. In this embodiment, the light chain is SEQ ID NO: 8. In yet a further embodiment, the antibody or fragment comprises a heavy chain amino acid sequence of SEQ ID NOS: 30, 26, 27, or 28. In another embodiment the antibody or fragment of any of the above heavy chains comprises a light chain of SEQ ID NO: 29. In still other embodiments, useful antibodies or fragment comprises three heavy chain CDRs from the heavy chain VH and full length heavy chain sequences of SEQ ID NO: 9, 11, 13, 15, 16, 19, 7, 23, 24, 25, 30, 26, 27, or 28. Light chain CDRs are obtained from light chains (VL or full sequences) of SEQ ID Nos: 10, 12, 14, 16, 18, 20, 22, 8 or 29.

The CDR1 sequences of variable heavy chains SEQ ID NOs 9, 11, 13, 15, 17, 19 or 22 are located at amino acid positions 31-35, respectively. The CDR2 sequences of variable heavy chains SEQ ID Nos: 9, 11, 13, 15, 17, 19 or 22 are located at positions 50-65, respectively. The CDR3 sequences of variable heavy chains SEQ ID Nos:9, 11, 13, 15, 17, 19 or 22 are located at positions 95-105, 95-104, 95-106, 95-104, 95-106, 95-105, and 95-107, respectively.

The CDR1 sequences of variable light chain sequences SEQ ID NO: 10, 12, 14, 16, 18, 20 and 22 are located at positions 24-34, 24-38, 24-34, 24-38, 24-40, 24-35, 24-39, respectively. The CDR2 sequences of variable light chain sequences SEQ ID NO: 10, 12, 14, 16, 18, 20 and 22 are located at positions 50-56, 54-61, 50-56, 54-61, 56-62, 51-57, and 55-61, respectively. The CDR3 sequences of variable light chain sequences SEQ ID NO: 10, 12, 14, 16, 18, 20 and 22 are located at positions 89-97, 94-101, 89-97, 93-100, 95-102, 91-97, and 95-102, respectively.

CDR1 of heavy chain SEQ ID NO: 7 is located at positions 40-45; CDR2 is located at positions 50-66; CDR3 is located at positions 99-109, respectively. CDR1 of light chain SEQ ID NO: 8 is located at positions 34-44; CDR2 is located at positions 50-56 and CDR3 is located at positions 89 to 97.

Still other LRRC15 antibodies useful in these methods are described in U.S. Pat. No. 10,188,660, European Patent No. EP3383909, published EP Application No. EP3383910A, US Patent Application publication Nos. 202000400672, 20190099431, 20190105329, and International Patent Application Publication No. WO2021/067673, incorporated herein by reference among others

Additional binding molecules useful in the methods herein include those molecules disclosed in US Patent Application publication 20050239700, incorporated herein by reference. Antibodies and/or binding fragments composing the anti-huLRRC15 antibodies generally comprise a heavy chain comprising a variable region (VH) having three complementarity determining regions (“CDRs”) referred to herein as VH CDR #1, VHCDR #2, and VH CDR #3, and a light chain comprising a variable region (VL) having three complementarity determining regions referred to herein as VL CDR #1, VL CDR #2, and VL CDR #3. The amino acid sequences of exemplary CDRs, as well as the amino acid sequence of the VH and VL regions of the heavy and light chains of exemplary anti-huLRRC15 antibodies and/or binding fragments are provided as previously described in U.S. Pat. No. 10,195,209, as well as others that can be readily obtained from commercial or institutional laboratories, or readily designed by conventional techniques. CDRs may be readily identified by methods known in the art including the Kabat or Chothia methods, described in detail in the website bioinf.org.uk/abs/info.html #cdrid, and by other algorithms known to the art. Specific embodiments of anti-huLRRC15 antibodies or binding fragments include, but are not limited to, those that include these exemplary CDRs and/or VH and/or VL sequences, as well as antibodies and/or binding fragments that compete for binding huLRRC15 with the exemplary antibodies and/or binding fragments. One example of an antibody and/or binding fragments composing the anti-huLRRC15 specifically binds huLRRC15 at a region of the extracellular domain (residues 22 to 527 of SEQ ID NO:3 of U.S. Pat. No. 10,195,209) that is shed from the cell surface and into the blood stream following cleavage at a proteolytic cleavage site (between residues Arg527 and Ser528 of SEQ ID NO:3 of U.S. Pat. No. 10,195,209). Still other antibodies identified in U.S. Pat. No. 10,195,209 are incorporated by reference herein.

Antibodies may be in the form of full-length antibodies, bispecific antibodies, dual variable domain antibodies, multiple chain or single chain antibodies, surrobodies (including surrogate light chain construct), single domain antibodies, camelized antibodies, scFv-Fc antibodies, and the like. They may be of, or derived from, any isotype, including, for example, IgA (e.g., IgA1 or IgA2), IgD, IgE, IgG (e.g., IgG1, IgG2, IgG3 or IgG4), IgM, or IgY. In some embodiments, the anti-huLRRC15 antibody is an IgG (e.g., IgG1, IgG2, IgG3 or IgG4). Antibodies may be of human or non-human origin. Examples of non-human origin include, but are not limited to, mammalian origin (e.g., simians, rodents, goats, and rabbits) or avian origin (e.g., chickens). In specific embodiments, antibodies are suitable for administration to humans, such as, for example, humanized antibodies and/or fully human antibodies.

Antibody antigen binding fragments composing the anti-huLRRC15 antibodies or fragments may include any fragment of an antibody capable of specifically binding huLRRC15. Specific examples of antibody antigen binding fragments that may be included in the anti-huLRRC15 ADCs include, but are not limited to, Fab, Fab′, (Fab′)2, Fv and scFv. Anti-huLRRC15 antibodies and/or binding fragments may include modifications and/or mutations that alter the properties of the antibodies and/or fragments, such as those that increase half-life and/or binding, etc., as is known in the art. In one embodiment, the LCCR15 antagonist is an antibody or antibody fragment that binds to one or more of an epitope of LCCR15. In another embodiment, the LCCR15 antagonist is an antibody or an antibody fragment which binds to two or more epitopes of LCCR15. In some embodiments, the LCCR15 antagonist binds to an epitope of LCCR15 such that binding of LCCR15 and its receptor are inhibited. In one embodiment, the epitope encompasses a component of a three-dimensional structure of LCCR15 that is displayed, such that the epitope is exposed on the surface of the folded LCCR15 molecule. In one embodiment, the epitope is a linear amino acid sequence from LCCR15.

For therapeutic uses, it is desirable to utilize anti-huLRRC15 antibodies or binding fragments that bind huLRRC15 with an affinity of at least 100 nM. Accordingly, in some embodiments, the anti-huLRRC15 comprise an anti-huLRRC15 antibody and/or anti-huLRRC15 binding fragment that binds huLRRC15 with an affinity of at least about 100 nM, or even higher, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, or greater affinity of anti-huLRRC15 antibodies and/or binding fragments can be determined using techniques well known in the art or described herein, such as for example, ELISA, isothermal titration calorimetry (ITC), surface plasmon resonance, flow cytometry, or fluorescent polarization assay.

Other non-antibody LCCR15 antagonists include antibody mimetics (e.g., Affibody® molecules, affilins, affitins, anticalins, avimers, Kunitz domain peptides, and monobodies) with LCCR15 protein or gene antagonist activity. This includes recombinant binding proteins comprising an ankyrin repeat domain that binds LCCR15 (protein or gene) and prevents it from binding to its receptor. The aforementioned non-antibody LCCR15 (protein or gene) antagonists may be modified to further improve their pharmacokinetic properties or bioavailability. For example, a non-antibody LCCR15 (protein or gene) antagonist may be chemically modified (e.g., pegylated) to extend its in vivo half-life. Alternatively, or in addition, it may be modified by glycosylation or the addition of further glycosylation sites not naturally present in the protein sequence of the natural protein from which the LCCR15 (protein or gene) antagonist was derived.

The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein.

The terms “RNA interference,” “RNAi,” “miRNA,” and “siRNA” refer to any method by which expression of a gene or gene product is decreased by introducing into a target cell one or more double-stranded RNAs, which are homologous to the gene of interest, LRRC15 (particularly to the messenger RNA of the gene of interest). Gene therapy, i.e., the manipulation of RNA or DNA using recombinant technology and/or treating disease by introducing modified RNA or modified DNA into cells via a number of widely known and experimental vectors, recombinant viruses and CRISPR technologies, may also be employed in delivering, via modified RNA or modified DNA, effective inhibition of LCCR15 to accomplish the outcomes described herein with the therapies described. Such genetic manipulation can also employ gene editing techniques such as CRISPR (Clustered Regularly Interspaced Short Palindromic Repeats) and TALEN (transcription activator-like effector genome modification), among others. See, for example, the textbook National Academies of Sciences, Engineering, and Medicine. 2017. Human Genome Editing: Science, Ethics, and Governance. Washington, D.C.: The National Academies Press. https://doi.org/10.17226/24623, incorporated by reference herein for details of such methods. In certain embodiments, siRNA sequences developed for mouse chondrocytes for assays using murine primary chondrocytes in vitro, as shown in the Table below. It is anticipated that similar sequences can be engineered for human samples. In one embodiment, human sequences having at least 50% sequence identity to the mouse sequences may also be used. In another embodiment, the human sequences may be less similar to the mouse sequences shown in the Table 1.

The following Table 1 identifies all of the sequences in the Sequence Listing Txt file associated with the application and incorporated by reference herein.

TABLE 1
Sequence Information

| SEQ | |
|---|---|
| ID NO | Sequence Referenced |
| 1 | Custom LRRC15 duplex cat # CTM-479162 mouse siRNA |
| | sense sequence |
| 2 | Custom LRRC15 duplex cat # CTM-479162 mouse siRNA |
| | antisense sequence |
| 3 | 581 amino acids short isoform of human LRRC15 protein |
| | (NP_570843.2) |
| 4 | 587 amino acid long isoform of human LRRC15 protein |
| | (NP_001128529.2) |
| 5 | 5881 nucleic acid sequence encoding SEQ ID NO: 3 |
| | (NM_130830.5) |
| 6 | 5938 nucleic acid sequence encoding SEQ ID NO: 4 |
| | (NM_001135057.3) |
| 7 | Heavy chain of anti-LRRX15 antibody (′209) |
| 8 | Light chain of anti-LRRX15 antibody (′209) |
| 9 | Heavy chain of variable region (VH) of anti-LRRX15 |
| | antibody (′209) |
| 10 | Light chain variable region (VL) of anti-LRRX15 |
| | antibody (′209) |
| 11 | Heavy chain (VH) of anti-LRRX15 antibody (′209) |
| 12 | Light chain (VL) of anti-LRRX15 antibody (′209) |
| 13 | Heavy chain (VH) of anti-LRRX15 antibody (′209) |
| 14 | Light chain (VL) of anti-LRRX15 antibody (′209) |
| 15 | Heavy chain (VH) of anti-LRRX15 antibody (′209) |
| 16 | Light chain (VL) of anti-LRRX15 antibody (′209) |
| 17 | Heavy chain (VH) of anti-LRRX15 antibody (′209) |
| 18 | Light chain (VL) of anti-LRRX15 antibody (′209) |
| 19 | Heavy chain (VH) of anti-LRRX15 antibody (′209) |
| 20 | Light chain (VL) of anti-LRRX15 antibody (′209) |
| 21 | Heavy chain (VH) of anti-LRRX15 antibody (′209) |
| 22 | Light chain (VL) of anti-LRRX15 antibody (′209) |
| 23 | Heavy chain of anti-LRRX15 antibody (′209) |
| 24 | Heavy chain of anti-LRRX15 antibody (′209) |
| 25 | Heavy chain of anti-LRRX15 antibody (′209) |
| 26 | Heavy chain of anti-LRRX15 antibody (′209) |
| 27 | Heavy chain of anti-LRRX15 antibody (′209) |
| 28 | Heavy chain of anti-LRRX15 antibody (′209) |
| 29 | Light chain of anti-LRRX15 antibody (′209) |
| 30 | Heavy chain of anti-LRRX15 antibody (′209) |

The term “small molecule” when applied to a pharmaceutical generally refers to a non-biologic, organic compound that affects a biologic process which has a relatively low molecular weight, below approximately 900 daltons. Small molecule drugs have an easily identifiable structure, that can be replicated synthetically with high confidence. In one embodiment a small molecule has a molecular weight below 550 daltons to increase the probability that the molecule is compatible with the human digestive system's intracellular absorption ability. Small molecule drugs are normally administered orally, as tablets. The term small molecule drug is used to contrast them with biologic drugs, which are relatively large molecules, such as peptides, proteins and recombinant protein fusions, frequently produced using a living organism.

The term “methylation modifying drugs” as used herein, and as an example of small molecules, enzymes and antisense nucleotides include drugs which affect chromatin architecture or DNA methylation. Such drugs include without limitation, hydralazine, isotretinoin, DNA methyltransferase (DNMT) 3a, DNMT3b, and DNMT1, 5-Azacytidine, Zebularine, Decitabine, the antisense oligonucleotide MG98, the small molecule RG108, FDCR, EGCG (see, e.g., Heerboth et al. Use of Epigenetic Drugs in Disease: An Overview. Genetics & Epigenetics 2014:6 9-19 doi:10.4137/GEG.S12270; and Lan Yi, et al., Selected drugs that inhibit DNA methylation can preferentially kill p53 deficient cells. 2014 October, Oncotarget. 5(19): 8924-8936).

Non-steroidal anti-inflammatory drugs include, but are not limited to, AMIGESIC® (salicylate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), and DAYPRO® (oxaprozin).

A “pharmaceutically acceptable excipient or carrier” refers to, without limitation, a diluent, adjuvant, excipient, auxiliary agent or vehicle with which an active agent of the present invention is administered. Pharmaceutically acceptable carriers are those approved by a regulatory agency of the Federal or a state government or listed in the U.S. Pharmacopeia or other generally recognized pharmacopeia for use in animals, and more particularly in humans, can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington. The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

By the term “nanocarrier” or “nanoparticle” is meant a submicron-sized colloidal systems (with a size below 1 μm), such as inorganic nanoparticles, lipidic, and polymeric nanocarriers carrier. Nanostructured delivery systems provide unique advantages, like protection from premature degradation and improved interaction with the biological environment. They also offer the possibility to enhance the absorption into a selected tissue, extend siRNA retention time, and improve cellular internalization. Such nanocarriers can comprise the selected inhibitor as a targeting moiety that directs the carrier to the local site of the OA. The targeting moiety may be a binding agent (e.g. the anti-LRRC15-antibody, an scFv fragment, or other antigen binding agent or a nucleic acid) that specifically recognizes the LRRC15 or its nucleic acid in the selected mammalian joint. In some embodiments, the LRRC15 inhibitor is enclosed within the carrier. In some embodiments, the selected inhibitor is covalently or non-covalently attached to the surface of the carrier. In some embodiments, the carrier is a liposome or a virus. Still other non-viral nanocarriers have been found useful for siRNA delivery. Nanostructured siRNA delivery systems include a wide variety of nanocarriers known in the art, such as lipid-based siRNA delivery systems, such as lumasiran and givosiran, as well as patisiran (Onpattro, Alnylam Pharmaceuticals) and some polymer-based siRNA delivery systems, such as siG12D-LODER. Polymeric nanocarriers can be prepared from different natural or synthetic polymers. Among polymer-based nanocarriers, those obtained from naturally occurring polysaccharides are highly biocompatible and non-immunogenic, including, without limitation, polysaccharidic nanocarriers based on chitosan and hyaluronic acid for small interfering RNA (siRNA) delivery. See, e.g., Serrano-Sevilla, I. et al., Natural Polysaccharides for siRNA Delivery: Nanocarriers Based on Chitosan, Hyaluronic Acid, and Their Derivatives, Molecules 2019 July; 24(14): 2570 PMID: 31311176; US Patent Publication No. 20200149026 and references cited therein, and Cuellar T L, et al. Systematic evaluation of antibody-mediated siRNA delivery using an industrial platform of THIOMAB-siRNA conjugates. Nucleic Acids Res. 2015; 43(2):1189-1203. doi:10.1093/nar/gku1362, incorporated by reference herein.

As used herein, the term “treatment” refers to any method used that imparts a benefit to the subject, i.e., which can alleviate, delay onset, reduce severity or incidence, or yield prophylaxis of one or more symptoms or progression of osteoarthritis. For the purposes of the present invention, treatment can be administered before, during, and/or after the onset of symptoms of osteoarthritis. In certain embodiments, treatment occurs after the subject has received conventional therapy. In some embodiments, the term “treating” includes abrogating, substantially inhibiting, slowing, or reversing the progression of advanced stages of osteoarthritis, substantially ameliorating, or substantially preventing the appearance of clinical or aesthetical symptoms of osteoarthritis, or decreasing the severity and/or frequency one or more symptoms resulting from OA.

As used herein, the term “prevent” refers to the prophylactic treatment of a subject who is at risk of developing progressively severe OA, resulting in a decrease in the probability that the subject will develop advanced stages of OA.

The terms “therapeutic effect” or “treatment benefit severity of OA”, as used herein mean an improvement in the health condition or diminution in severity of OA, for example, a decrease in pain, an increase in mobility or flexibility of the joint, or an improvement or diminution in severity of conventional treatment side effect.

A “therapeutically effective amount” of a compound or a pharmaceutical composition refers to an amount effective to prevent, inhibit, treat, or lessen the symptoms and/or progression of osteoarthritis. An “effective amount” is meant the amount of LRRC15 antagonist composition sufficient to provide a therapeutic benefit or therapeutic effect after a suitable course of administration. It should be understood that the “effective amount” for the composition which comprises the LRRC15 antagonist vary depending upon the inhibitor/antagonist selected for use in the method. Regarding doses, it should be understood that “small molecule” drugs are typically dosed in fixed dosages rather than on a mg/kg basis. With an injectable, a physician or nurse can inject a calculated amount by filling a syringe from a vial with this amount. In contrast, tablets come in fixed dosage forms. Some dose ranging studies with small molecules use mg/kg, but other dosages can be used by one of skill in the art, based on the teachings of this specification.

The “effective amount” for a protein or peptide antagonist, e.g., antibody, antibody fragment or recombinant protein or peptide, the effective amount can be about 0.01 to 25 mg antibody/injection. In one embodiment, the effective amount is 0.01 to 10 mg antibody/injection. In another embodiment, the effective amount is 0.01 to 1 mg antibody/injection. In another embodiment, the effective amount is 0.01 to 0.10 mg antibody/injection. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg antibody/injection. Still other doses falling within these ranges are expected to be useful. In one embodiment an effective amount for the nucleic acid and/or protein inhibitor of composition (a) includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 1 to 20 mg/kg body weight. Still other doses falling within these ranges are expected to be useful.

The term “therapeutic regimen” as used herein refers to the specific order, timing, duration, routes and intervals between administration of one of more therapeutic agents or antagonists. In one embodiment a therapeutic regimen is subject-specific. In another embodiment, a therapeutic regimen is disease stage specific. In another embodiment, the therapeutic regimen changes as the subject responds to the therapy. In another embodiment, the therapeutic regimen is fixed until certain therapeutic milestones are met. In one embodiment of the methods described herein, the administration of a composition that blocks or inhibits the expression, induction, activity, or signaling of LCCR15 (protein or gene) involves one or more doses of the same composition or one or more doses of different antagonist compositions.

Once the subject is evaluated and the OA is under control, not increasing in severity or preferably decreasing in severity as judged by physical examinations, the therapeutic regimen may be adjusted for maintenance of improvement by maintaining the LRRC15 antagonist doses. Alternatively, the LRRC15 antagonist can be administered less frequently but for a longer duration. In one embodiment, the dose and dosage regimen of the that is suitable for administration to a particular patient may be determined by a physician considering the patient's age, sex, weight, general medical condition, and the stage and severity of the OA. The physician may also consider the route of administration of the agent, the pharmaceutical carrier with which the agents may be combined, and the agents' biological activity. Additionally, the LRRC15 antagonist may be co-administered with other appropriate therapies for OA.

By “administration” or “routes of administration” include any known route of administration that is suitable to the selected inhibitor or composition, and that can deliver an effective amount to the subject. In one embodiment of the methods described herein, the routes of administration include one or more of oral, parenteral, intravenous, intra-nasal, sublingual, by inhalation or by injection directly into the site of the OA.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

### Pharmaceutical Preparations

In one embodiment a single composition comprises at least one anti-LRRC15 antibody or antibody fragment and at least one carrier (e.g., pharmaceutically acceptable carrier). In another embodiment, a single composition comprises at least two anti-LRRC15 antibodies or antibody fragments and at least one carrier (e.g., pharmaceutically acceptable carrier). In another embodiment a single composition comprises at least one anti-LRRC15 nucleic acid sequence, such as an siRNA, and at least one carrier (e.g., pharmaceutically acceptable carrier).

The pharmaceutical preparations containing the anti-LRRC15 antibodies or LRRC15-antagonizing nucleic acid sequences, small molecules or any of the other components identified above may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the inhibitors or compositions to be administered, its use in the pharmaceutical preparation is contemplated.

In one embodiment, the pharmaceutical preparations containing the anti-LRRC15 antibodies or LRRC15-antagonizing nucleic acid sequences composition are associated with nanocarriers as described above. In one embodiment, such a nanocarrier associated composition is suitable for local delivery to the OA-affected joint or site. In one embodiment, the composition includes an LRRC15 siRNA or antagonist and/or nanocarrier-based siRNA conjugated to anti-LRRC15 antibody for more efficient delivery with dual effect of siRNA/antagonist and antibody. Methods for the design of such compositions can be found in Serrano-Sevilla I et al 2019, and/or Cuellar T L et al 2014, described above.

In another aspect, the pharmaceutical composition can be comprised of small peptides that are tested for effective LRRC15 blockade by specifically targeting methylation motifs of LRRC15. Such compositions can be designed in a manner similar to that described in Gayatri S, et al. Using oriented peptide array libraries to evaluate methylarginine-specific antibodies and arginine methyltransferase substrate motifs. Sci Rep. 2016 June; 6:28718. doi:10.1038/srep28718, incorporated by reference herein.

Selection of a suitable pharmaceutical preparation depends upon the method of administration chosen. For example, the composition may be administered by direct injection into the affected joint. In this instance, a pharmaceutical preparation comprises the agents dispersed in a medium that is compatible with intra-articular delivery. Pharmaceutical agents may also be administered parenterally by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the agents, or the pharmaceutical preparation in which they are delivered, may be increased so that the molecules can better arrive at their target locations.

Pharmaceutical compositions containing the LRRC15 gene or LRRC15 protein inhibitors and/or antagonists as the active ingredient in intimate admixture with a pharmaceutical carrier can be prepared according to conventional pharmaceutical compounding techniques. The carrier may take a wide variety of forms depending on the form of preparation desired for administration, e.g., local for injection into the joint or site of OA (see e.g., US Patent Publication No. 20200149026) or systemic. For example, in preparing the agent in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like. For parenteral compositions, the carrier will usually comprise sterile water, though other ingredients, for example, to aid solubility or for preservative purposes, may be included. However, the local injectable suspensions may also be prepared, in which case appropriate liquid carriers, suspending agents and the like may be employed as described above.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the compositions of the invention may be determined by evaluating the toxicity of the active therapeutic inhibitor in animal models. Various concentrations of the above-mentioned inhibitors including those in combination may be administered to a mouse model of OA, and the minimal and maximal dosages may be determined based on the results of significant reduction of pain and increase in mobility/flexibility without significant side effects as a result of the treatment.

In one embodiment, these compositions can also include adjunctive therapeutics including, without limitation, anti-inflammatory drugs. In one embodiment, these compositions are designed for local administration and include such adjunctive therapeutics such as anti-inflammatory drugs for local delivery, e.g., to the arthritic joint in question. In another embodiment, these compositions include upstream modulators of LRRC15 expression, such as, IL-1β, TNF-α, certain MAP kinases, and members of the NFκB signaling pathway. In yet other embodiments, these compositions include small molecule inhibitors of LRRC15 protein activity or LRRC15 gene expression.

The compositions comprising the LRRC15 gene or LRRC15 protein antagonists of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Diagnostic Methods

Another aspect of the present invention is a method of diagnosing early stage osteoarthritis by detecting levels of LRRC15 protein and/or detecting levels of methylation of the LRRC15 gene. As noted in the examples and specification, detection of LRRC15 may be used as a means for diagnosis of early-stage osteoarthritis. The method includes measuring the level of LRRC15 protein in a sample from a subject. In one embodiment, the sample is synovial fluid. In another embodiment, the sample is PBMC. In another embodiment, the sample is cartilage or bone tissue. In some embodiments, the level of LRRC15 is detected in a sample obtained from a subject. This level may be compared to the level of a control. “Control” or “control level” as used herein refers to the source of the reference value for LRRC15 levels. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom.

The antibodies and LRRC15 antagonists described above may be used in such diagnostic methods to diagnose early-stage osteoarthritis using conventional diagnostic labels and reagents. Additional methods for diagnosis include detecting the levels of methylation and demethylation of the LRRC15, wherein detection of significant 5 methyl cytosine hypomethylation indicates early-stage osteoarthritic cartilage. An increase in the level of LRRC15 protein indicates early-stage OA or progressive OA. The diagnostic method may also be employed in a method of assessing the efficacy of a treatment for OA by obtaining a baseline level of LRRC15 protein from the subject prior to, or at the beginning of treatment for OA. After a desirable time period, the level of LRRC15 protein in the subject is measured again. A decrease in the level of LRRC15 protein as compared to the earlier time point indicates that the treatment for the OA or fibrosis is, at least partially, efficacious. The treatment may be any of those described herein, or other treatments deemed suitable by the health care provider.

In still another embodiment, the diagnostic method may further include a step of treating the subject for osteoarthritis, by the means discussed below.

### Methods of Treatment

The primary purpose of these methods is to target the abnormal LRRC15 expression and/or activity observed in cartilage and other OA joint tissues aiming to prevent the OA development and/or progression.

In one aspect, a method of treating or reducing the progression of osteoarthritis (OA) comprises administering to a subject having OA an effective amount of a composition that blocks, antagonizes or inhibits the expression, induction, activity, methylation, or signaling of the LRRC15 gene or binds, blocks, antagonizes or inhibits the activity or signaling of LRRC15 protein in vivo. One embodiment of this method involves administering to a human having OA an effective amount of at least one compound, construct or composition that specifically binds to human LRRC15 protein. Another embodiment of this method involves administering to a human having OA an effective amount of at least one compound, construct or composition that inhibits the transcription, expression or activity of the LRRC15 gene or modifies or silences the expression of the LRRC15 protein in vivo.

As described above, for inhibiting the transcription, expression or activity of LRRC15 gene or modifies or silences the expression of LRRC15 protein in vivo, the method can employ an RNA or DNA construct that inhibits the expression of LRRC15. In one embodiment, the construct comprises a nucleic acid molecule that inhibits the translation or transcription of LRRC15 gene. For example, a human may be administered an effective amount of a recombinant virus or virus-like particle that expresses an LRRC15 antagonist. In another embodiment, a human patient may be administered a DNA construct that expresses an LRRC15 antagonist in vivo. In another embodiment, the patient is administered an siRNA or shRNA sequence to interfere with transcription or activity of the gene. In yet another embodiment, a CRISPR construct is designed to interrupt or modify expression, transcription or activity of the LRRC15 in vivo so that the gene cannot operate normally.

In still other embodiments, a patient is administered a composition comprising an LRRC15 antagonist as a peptide or protein, an antibody or antigen-binding fragment that specifically binds to and inhibits the activity of LRRC15 protein in vivo.

In other embodiments, a patient is a small molecule inhibitor that targets LRRC15 gene or protein directly, or a salt, enantiomer or prodrug thereof.

In any of these embodiments of the method of treatment, the composition being administered further comprises a pharmaceutically acceptable excipient or carrier. In still other embodiments, the methods involve additional adjunctive treatment steps for OA including administering anti-inflammatory drugs. In one embodiment, these adjunctive therapies include anti-inflammatory drugs for local delivery, e.g., to the arthritic joint in question. Concomitant administration of LRRC15 with anti-inflammatory compounds is likely to be beneficial; in one embodiment, such administration is local to the joint in question. In another embodiment, these therapies include co-administering to the subject, either with the antibodies or in a separate administration step, certain upstream modulators of LRRC15 expression, such as, IL-1β, TNF-α, and certain MAP kinases. In yet other embodiments, small molecule inhibitors of LRRC15 activity or LRRC15 expression may be administered as adjunctive therapies with the antibodies discussed herein. In one embodiment, such adjunctive therapies are administered by the same route or administration as the antibodies or in different routes of administration according to a designated therapeutic regimen.

Whether the treatment of the patient having OA symptoms involves nucleic acid components or protein/components or even small molecules, the methods may involve administering the compositions in a single dose or as one or more booster doses. In one embodiment, the method involves intra-articular injection to deliver the composition to the site of the joint with OA damage. In other embodiments, the composition is administered systemically by oral, intramuscular, intraperitoneal, intravenous, intra-nasal administration, sublingual administration or intranodal administration or by infusion.

In yet a further embodiment, a method of treating an arthritic joint comprising injecting into the joint of a mammalian subject having osteoarthritis an effective amount of a composition that blocks, antagonizes or inhibits the expression, induction, activity, methylation, of the LRRC15 gene or binds, blocks, antagonizes or inhibits the activity of LRRC15 protein in vivo. In one embodiment, the method is administered to a human subject to treat or retard the progression of OA. The stage of OA can be early or advanced, and it is anticipated that this treatment would be effective.

In addition to the methods outlined above, the (a) modification of LRRC15 gene expression can be achieved by genomic and epigenomic editing, or delivery of methylation modifying drugs; and (b) modification of LRRC15 protein activity can be achieved by delivery of small molecule inhibitors, or nanoparticles conjugated with antibodies/small molecule inhibitors against LRRC15. Targeting LRRC15 will dampen the abnormal activation of a number of catabolic genes that contribute to tissue destruction in OA, without impacting molecules involved in anabolism/homeostasis. Given that we will target a gene that is abnormally expressed in pathological conditions, we do not expect an impact in normal tissue remodeling or cellular homeostasis.

The methods and compositions of this invention apply the observations set out in detail in the examples below. To dissect changes in DNA methylation with a functional impact that occur during OA progression, we used the destabilization of the medial meniscus (DMM) surgical model to identify temporal changes in DNA methylation patterns associated with structural and transcriptomic changes in cartilage during osteoarthritis (OA) progression. The DMM model mimics human post-traumatic OA driven by meniscal injury and has been successfully used by our lab and others to understand progressive changes in OA disease, and to demonstrate the importance of aggrecan- and collagen-degrading enzymes, kinases, and transcription factors in cartilage destruction.

Combining the surgical model of OA with transcriptomic and epigenomic analyses, and with work with human and murine OA cartilage, and in vitro models using human and primary chondrocytes, here we show that the progression of OA is accompanied by dynamic, time-dependent changes in DNA methylation patterns. Integrating our transcriptomic and epigenomic datasets along with comparing with human data set, we identified the novel gene LRRC15 as one of the genes differentially methylated and expressed in early OA cartilage, and we show that LRRC15 contributes to the IL-1β-driven expression of OA relevant catabolic genes in primary chondrocytes in vitro. Together, our findings further support the contribution of DNA methylation to OA disease, highlight the need of dissecting early and late-stage disease phases given the dynamic nature of these changes and the potential changes driven by cartilage loss, and show that such integrative analyses have the potential of uncover novel targets with therapeutic potential that participate in the early phases of the disease.

In the examples, the inventors used a well-established mouse model of surgically induced post-traumatic OA (PTOA) to capture changes in gene expression and DNA methylation that occur during the progression of OA disease. Our integrative analyses and the comparison with human datasets led to the identification of time-dependent epigenomic signatures that overlap with changes in gene expression during the progression of OA. Notably, we identify LRRC15 as a novel gene that contributes to OA disease and displays methylation-sensitive changes in gene expression.

Additionally, our gene analysis in early and advanced OA demonstrated that in early OA, 2 genes at 4 weeks and 31 genes at 12 weeks including extracellular matrix genes or genes associated with ECM like LRRC15, Aspn, Col5a1, Col6a3, Tns1, C1qtnf1, Antxr1 membrane transporters Slc16a2, Slc35e4, phosphatases like ptpn14 and metalloproteases Adamtsl5, timp2 were differentially expressed and associated with changes in their methylation status. Pathway analysis identified 33 GO biological processes that involves changes in gene expression and 12 BP that involves phenotypic changes and 6 BP that involves phenotypic changes that are associated with gene expression. These pathways are similar to that were reported earlier to be crucial for OA progression in human and PTOA model in mice (Ji Q et al 2019; Sebastian A et al 2018). This data indicates that progression of OA requires continuous epigenetic and transcriptional changes to facilitate disease progression.

On comparison of our dataset with human orthologs in OA, we used HuGENet and recent publications that used human OA samples for RNA seq and or methylation analysis. Knowing the fact that all the studies are performed using different criteria of sample selection, sequencing methods and different analysis parameter, we divided published data is into two simple categories—comparing OA to healthy control and eroded cartilage, that may contain subchondral bone to intact cartilage. Out of 168 genes implicated in OA in HuGENet, 28 genes overlapped with our data, 19 genes with DEGs and 9 genes with DMR. On splitting 28 genes between 4 and 12 weeks timepoints 6 overlapping DEGs and 2 DMR were identified and at 12 weeks 13 overlapping DEGs and 7 DMRs were identified. Asporin emerged as the gene that is differentially expressed and methylated in human and in mouse at 12 weeks.

Comparison with OA vs healthy controls using four published data set 248 human DEGs and 10 DMR overlapped with our data set, some of the genes that overlapped with differential expression in human are PTGS2, ASPN, RUNX1, LRRC15, Lrrc17, CXCL14, metalloproteases MMP19 and MMP2, extracellular matrix proteins like Col14A1, Col4A1, Col12A1, Col3A1, Col6A1, Col6A2, Col5A1, COMP MAMDC2. MAMDC2 is also reported earlier to be upregulated in PTOA model (Karlsson C et al 2010, Fernandez T J et al 2014, Chen L et al 2018 and Chen Y J et al 2018, Sebastian A et al 2018; Steinberg et al 2017). 10 genes that overlapped with human methylation data are ARAP1, FZD9, HTRA4, IGSF9, Il11RA, RUNX1, S100A10, SKAP1, TNS1, WiPF1. Runx 1 was only gene that was differentially expressed and methylated in humans as seen in our data set at 12 weeks (Karlsson C et al 2010, Fernandez T J et al 2014, Chen L et al 2018 and Chen Y J et al 2018). Similarly, comparison with eroded cartilage vs intact cartilage showed 618 overlapping genes comprised of transcription factors, cytokines, metalloproteinases, metallopeptidases and various collagens (Jeffries M A 2014, 2016, Dunn S L et al 2016, Steinberg et al 2017, Liu Y et al 2018, Li H et al 2019; and data not shown). Single cell RNA seq analysis of OA chondrocytes isolated from OA patients revealed 4 genes predictive of OA-ADRM1, HSPA2, RPS29 and Col5a1, out of these 4 genes Col5a1 overlaps with our dataset is differentially expressed at both 4 and 12 weeks and differentially methylated at 12 weeks (Ji Q et al 2019).

Comparing human data with our data suggests that PTOA mouse model can be used to study OA progression and to identify potential biomarkers that are predictor or targets for OA. Another interesting observation of this analysis reveals that no two studies have identical data sets. There are overlaps but the individual sets are still unique to each study depending on sample selection criteria sequencing and analysis approach, suggesting that OA is a systemic disease and several factors affects its progression and at changes in gene expression at molecular levels (Soul et al 2019).

One of the interesting observations we made was LRRC15 was upregulated in human OA samples and it happens to be the only gene at 4 weeks that was most expressed and inversely associated with methylation at early stage of OA (Chen L et al 2018, Ji Q et al 2019; Chen Y J et al 2018). LRRC15 continues to be differentially expressed, but not differentially methylated at 12 weeks. One of the reasons for this inconsistency could be attributed to increased erosion of cartilage at later time point. Based on our observation and other reports LRRC15 likely contributes to phenotypic dysregulation of articular chondrocytes.

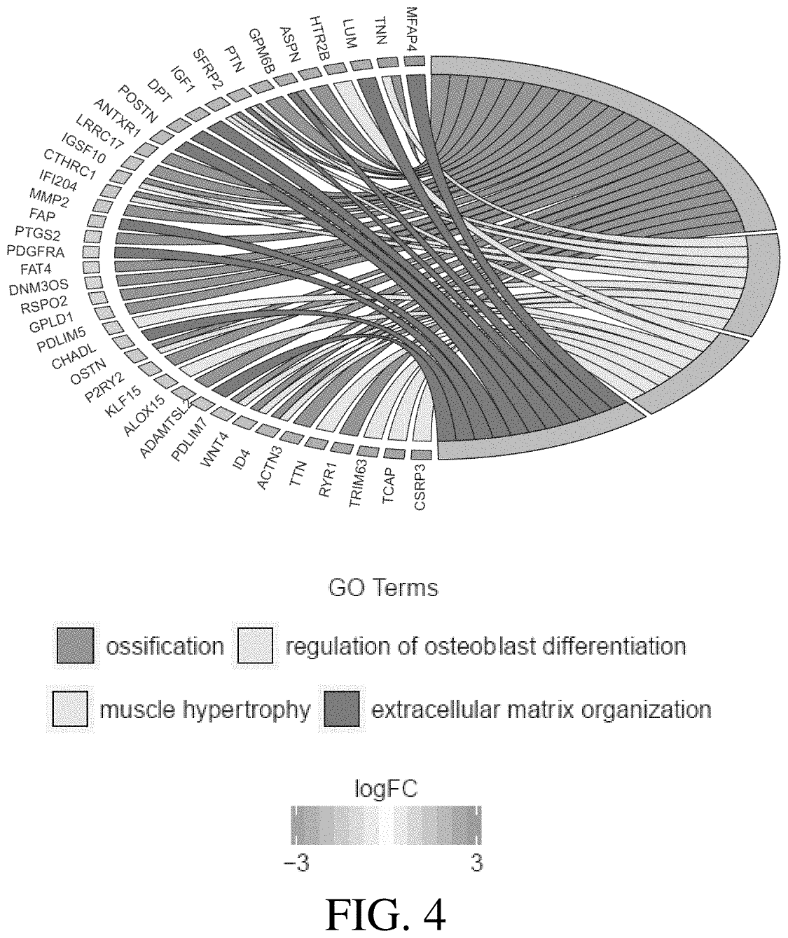

LRRC15 is leucine rich transmembrane protein, also known as lib and is conserved from Drosophila to humans, it consists of an extracellular domain, transmembrane domain and a very short cytoplasmic domain and because of its structural similarity it has been clustered together with toll like receptors and other LRR genes (Dolan J et al 2007). Proinflammatory cytokines upregulate LRRC15 expression as indicated by our data (See also, FIG. 4; Satoh K et al 2002). In normal tissue, during development its expression is localized to invasive cytotrophoblast in placenta and hypertrophic zone in mouse growth plate (Reynold P A et al 2003; unpublished data). Our immunohistochemistry data shows LRRC15 is localized to calcified lesions. In support of our finding, other have also reported LRRC15 upregulated expression in human osteoarthritis and in osteoclast in RA (Chen L et al 2018, Ji Q et al 2019; Chen Y J et al 2018). All these evidences suggest that LRRC15 might be involved in calcification and osteophyte formation that are hallmark features of advanced OA.

Although not much is known about the mechanism of LRRC15 functions, one report has shown LRRC15 negatively regulates NF-κB pathway to promote osteogenesis by inhibiting p65 nuclear translocation (Wang Y et al 2018). On the contrary, NF-κB pathway is one of the major pathway that transmits signals triggered by the inflammatory factors, that leads to increased catabolic activity causing ECM degradation and cartilage damage (Marcu K B et al 2010; Roman-Blas J A et al 2006; Saklatvala J et al 2007; Goldring M et al 2009). We observed LRRC15 dependent upregulation of catabolic genes like MMP13; Cox2 and Elf3. We suspect that LRRC15 functions through regulation by, and interaction with, the NF-κB pathway

Together, the results here presented show that dynamic changes in the DNA methylation patterns of articular cartilage take place during OA disease progression. These dynamic changes may have a functional impact and contribute to the expression of genes abnormally regulated in the early disease stages, like LRRC15, which in turn can alter the phenotype and responses of OA chondrocytes, thus contributing to the disease onset and progression.

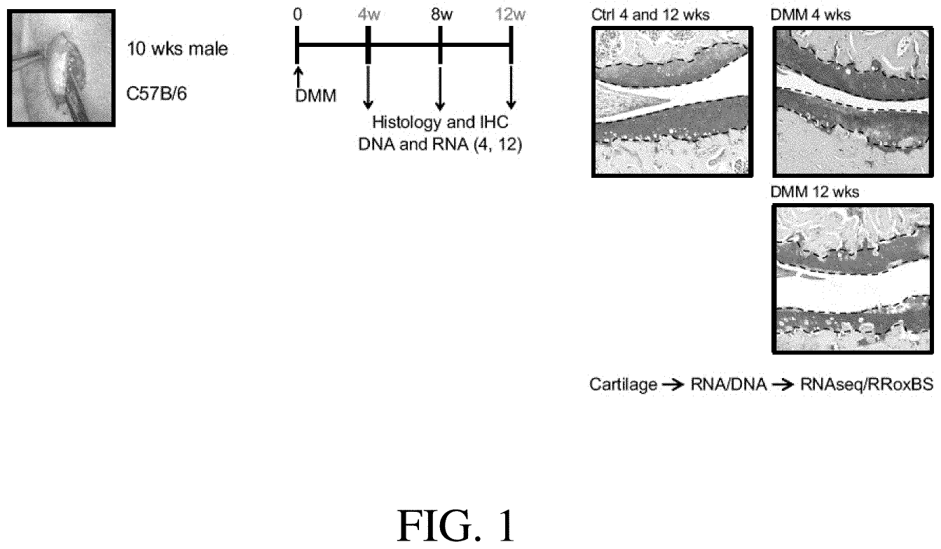

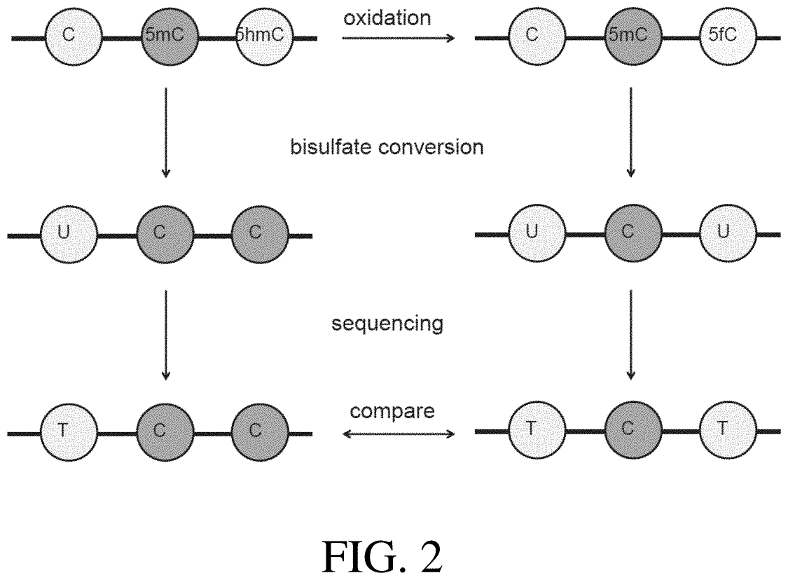

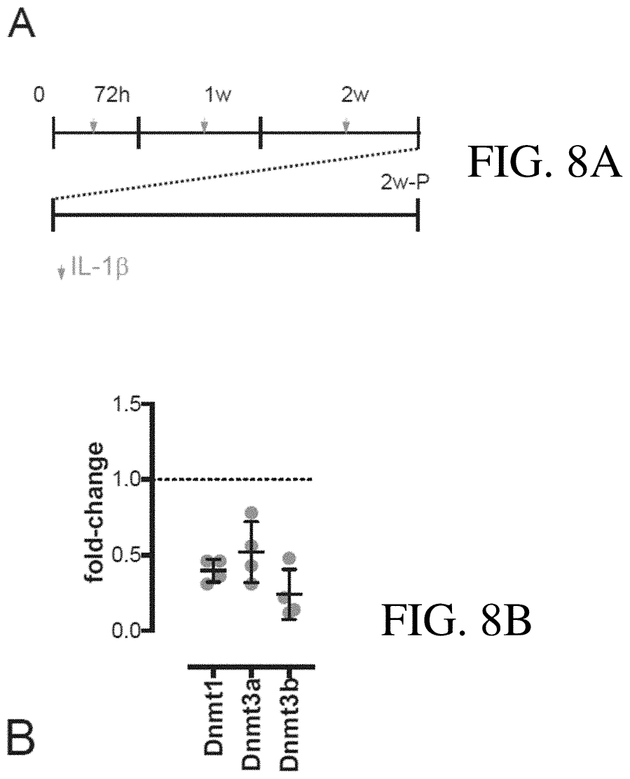

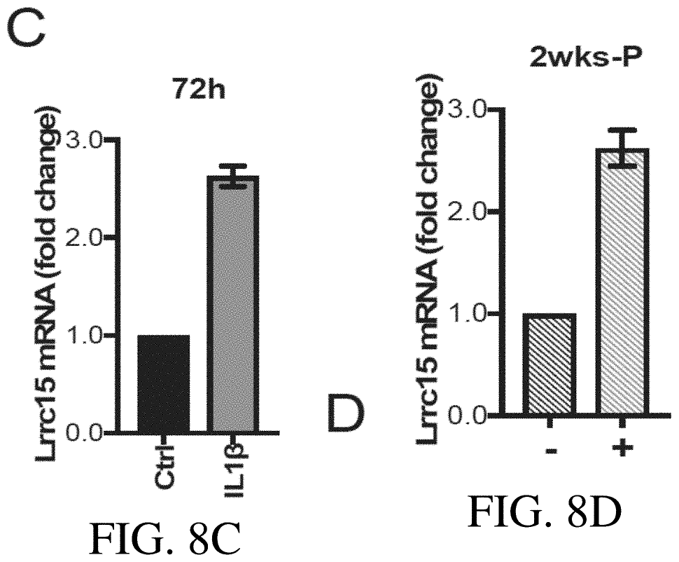

As demonstrated in the examples below and the attached FIGS. 1-9, the inventors identified time-dependent alterations in epigenomic patterns in cartilage after DMM, with significant changes in 5 mC and 5 hmC methylation comparing samples retrieved at 4 and 12 weeks after surgery. Integration of RNAseq and RRoxBS datasets identified LRRC15 among the hypomethylated genes with increased expression at 4 weeks after surgery. We confirmed LRRC15 immunostaining in human and murine OA cartilage, and experiments in human and murine primary chondrocytes showed that the expression of LRRC15 is DNA methylation-dependent and induced by IL1β and TNFα. Knockdown experiments showed that LRRC15 contributes to the IL1β-driven expression of catabolic genes relevant to OA, including Mmp13.

### Example 1: Methods

RNA sequencing (RNAseq) and Reduced Representation Oxidative Bisulfite Sequencing (RRoxBS) analyses were done in total RNA and DNA obtained from micro-dissected cartilage after DMM. Murine and human primary chondrocytes were used to evaluate the cytokine- and methylation-dependent changes in the expression of LRRC15, and its contribution to IL-1β-induced changes in chondrocytes.

Statistical analyses were performed using GraphPad Prism 7 Software (GraphPad Software, Sand Diego, Calif.) and subsequently by GraphPad Prism 8 Software. Data are reported as means±S.D. or as median and 95% C.I. (histological scores) of at least three independent experiments. Unpaired Student t-test was used to establish statistical significance between two groups. Analysis of the histological scores was performed using Mann-Whitney test. For data involving multiple groups, one-way analysis of variance (ANOVA) was performed followed by Tukey's post-hoc test. P<0.05 was considered significant.

### Example 2: Epigenomics and Transcriptomics Analyses that Uncovered LRRC15 as a Differentially Methylated and Expressed Gene in Early OA Cartilage

To identify early changes in DNA methylation with a functional impact in gene expression and disease progression, we used the destabilization of the medial meniscus (DMM) model of post-traumatic OA³, which mimics post-traumatic OA in humans, paired with epigenomic (DNA methylation analyses, using RRoxBS) and transcriptomic (RNA-seq) analyses in cartilage obtained at 4 (early OA) and 12 weeks (established OA) after DMM surgery. We identified temporal changes in DNA methylation patterns that are associated with transcriptomic and structural changes in OA cartilage. This assay can also be used to identify other genes that contribute to the dysregulated phenotype of OA chondrocytes and to OA progression.

DMM surgeries were performed in weight-matched 10 week old male C57BL/6J mice. The left knees were used as unoperated controls. Articular cartilage was micro-dissected and used for RNA and DNA isolation at 4 and 12 weeks after surgery. Total RNA was used for RNA sequencing, and DNA was used for Reduced Representation Oxidative Bisulfite Sequencing (RRoxBS). RNAseq reads were processed using a dedicated RNAseq pipeline. Changes in selected differentially expressed genes were further validated using SYBR-green based real-time PCR analyses.

For methylation profiling, per sample, 50-60 million RRBS reads were aligned and processed using a bioinformatics pipeline to yield methylation values for each CpG. Oxidative bisulfite (oxBS) technology was applied to distinguish between 5 mC and 5 hmC. Methylation values at the CpG sites assayed by RRoxBS were interrogated for significant differences (q<0.05 and methylation difference of at least 25%) using the Bioconductor R package methyl Kit. The site-specific differential methylation data was then queried for differentially methylated regions (DMRs) using the Bioconductor R package eDMR.

Histological and Immunohistochemical assays were used to evaluate cartilage degradation and the presence of LRRC15 protein. In vitro assays using murine and human primary chondrocytes were used to further evaluate the cytokine- and methylation-dependent changes in the expression of LRRC15. siRNA-mediated knockdown experiments were used to study the contribution of LRRC15 to the IL-1β-induced changes of Mmp13 in articular chondrocytes.

Histological scoring confirmed the time-dependent progression of OA after DMM. RNAseq data comparisons between OA and control samples uncovered 529 differentially expressed genes (DEGs) at 4 weeks post-DMM, and 589 DEGs by 12 weeks after surgery. Several DEGs unique to early (4 weeks) and established (12 weeks) OA were identified, along with overlapping DEGs. RRoxBS analyses revealed significant differences in DNA methylation between control and surgical groups at both 4 and 12 weeks. The number of differentially methylated 5 mCs and 5 hmCs dramatically increased from 4 to 12 weeks after DMM. Unique differentially methylated genes were identified for early and established OA. Correlative analyses of RRoxBS and RNAseq data identified genes that are differentially methylated and differentially expressed. The leucine-rich repeat containing 15 (LRRC15) gene was a hypomethylated gene with increased expression at 4 weeks after DMM.

We confirmed LRRC15 immunostaining in OA cartilage samples, and IL1β- and TNFα-induced expression of LRRC15 in chondrocytes. Treatment with the DNA methyl transferase inhibitor (5-aza-deoxycytidine) lead to increased LRRC15 mRNA in vitro, confirming the methylation-dependent expression of LRRC15 in chondrocytes. LRRC15 knockdown experiments showed that LRRC15 contributes, at least in part, to the IL1 τ3-driven expression of catabolic genes relevant to OA, including Mmp13. Here, we show that the progression of PTOA in the DMM model is accompanied by dynamic CpG methylation changes in cartilage, and that the changes in DNA methylation patterns are time-dependent and associated with transcriptomic changes. Our data further highlight the contribution of changes in DNA methylation to the altered phenotype and gene expression of OA articular chondrocytes. In addition, our integrative analyses uncovered that the novel LRRC15 gene is differentially methylated and expressed in early OA disease, and that it may contribute to the phenotypic dysregulation of articular chondrocytes in OA dis1.

This and additional experiments demonstrate that changes in structure and gene expression are associated to time dependent changes in DNA methylation patterns in articular cartilage in the progression of OA after DMM surgeries. Abnormal methylation explains changes in LRRC15 expression in articular chondrocytes in vivo and in vitro. Additional examples will demonstrate the functional contribution of LRRC15 to cartilage homeostasis and osteoarthritis and identify mechanistic connections between changes in DNA methylation and the expression of other genes relevant to OA. Thus, LRRC15 can be targeted therapeutically in the treatment of OA.

Together, the results of Example 1 and 2 show changes in LRRC15 gene expression and DNA methylation in early OA, and that LRRC15 contributes to the expression of genes known to contribute to OA disease in vitro. Thus, modulation of LRRC15 expression and/or activity in vivo is likely therapeutic strategy in OA.

### Example 3—The Progression of Osteoarthritis after DMM Surgery is Accompanied by Time-Dependent Transcriptional Changes in Articular Cartilage

To evaluate how the gradual changes in chondrocytes associate with disease progression and to evaluate genomics changes during progression of OA, we undertook an integrative approach whereby we analyzed a) cartilage structural damage using histological approaches, b) changes in gene expression occurring over time using RNAseq, and c) progressive time-dependent alterations in 5 mC and 5 hmC DNA methylation patterns by RRoxBS. These analyses were performed in cartilage samples retrieved at 4 and 12 weeks after DMM.

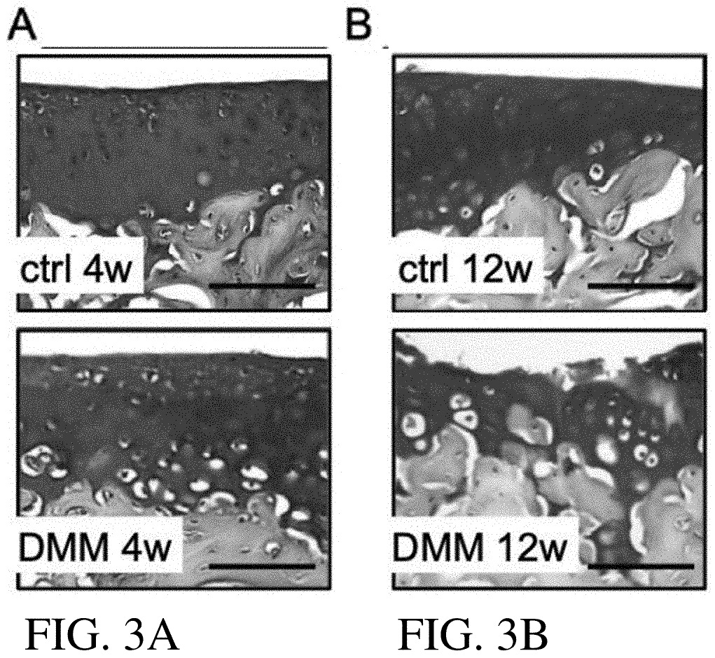

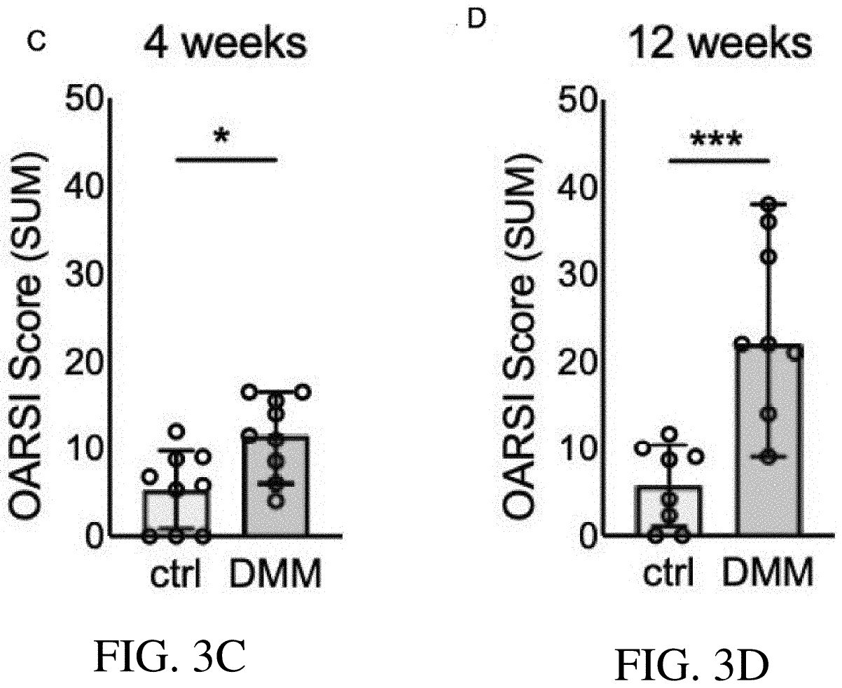

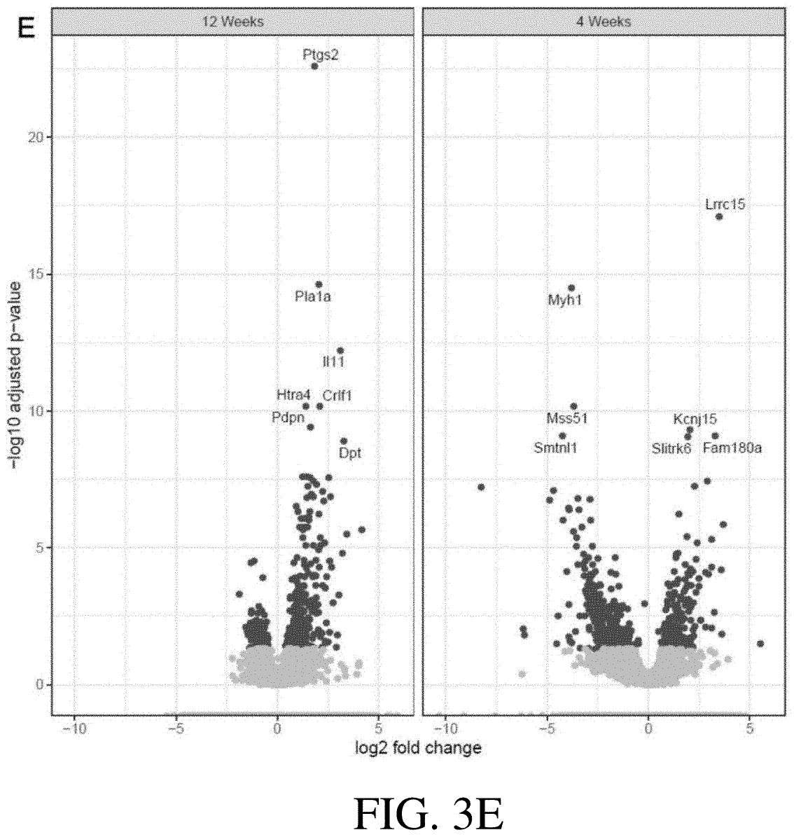

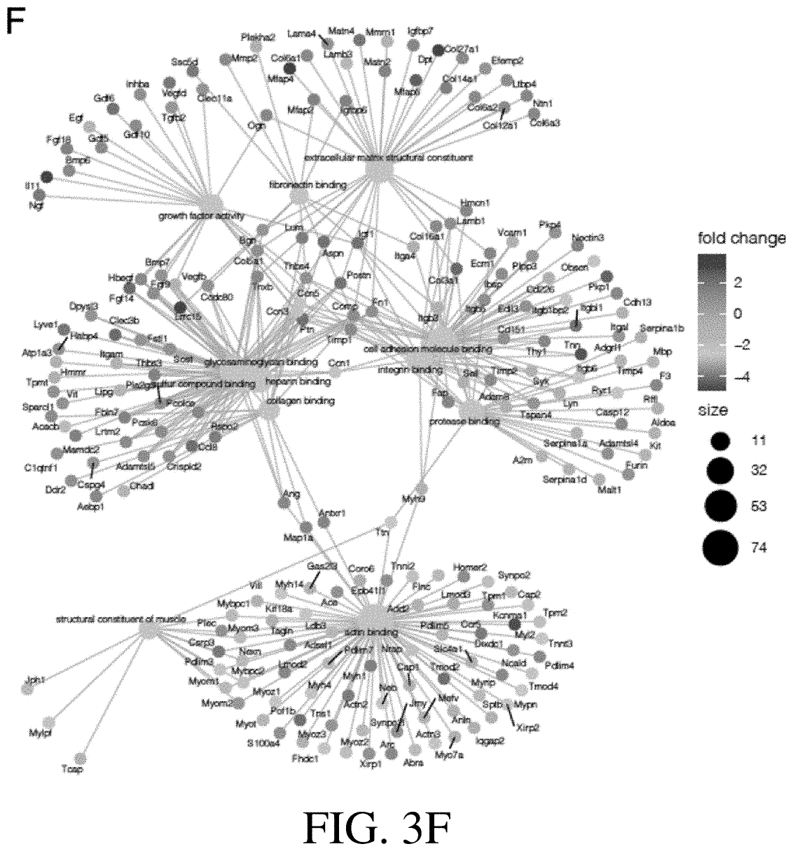

To confirm the progression of OA after DMM, we evaluated tissues histologically. As shown in FIGS. 3A and 3B, respectively, the initial loss of proteoglycan staining and minor surface damage at 4 weeks was followed by the more evident fibrillation and structural changes in tissues collected at 12 weeks after surgery. These progressive structural changes were also evident and confirmed in the OARSI histological SUM scores (FIG. 3C-3). The contralateral, control legs showed no changes, as expected (data not shown).

We next evaluated changes in gene expression occurring in articular cartilage during the progression of OA using RNAseq in total RNA isolated from microdissected cartilage tissues collected at 4 and 12 weeks after DMM surgery. Comparing DMM-operated (n=3 per time-point) and control, non-operated limbs (3=3 per time point) from the same mice, we identified 529 and 589 differentially expressed genes (differentially expressed genes ((DEGs), Benjamini-Hochberg (BH) adjusted p-value <0.05)) at 4 and 12 weeks after DMM, respectively (data not shown). Comparison of differentially expressed genes (DEGS) at 4 and 12 weeks identified 474 genes unique to early OA (4 weeks), 528 genes unique to more established OA cartilage (12 weeks), and 55 DEGs common to both 4 and 12 weeks. In addition to uncovering novel genes with potential relevance to the early phases of OA disease (including LRRC15 or Lrrc17), our RNAseq analyses confirmed previous reports showing changes in the expression of genes with known contribution to OA, including Aspn, Adamts16, Mmp3 and Ptgs2 (data not shown and Loeser R F et al 2013; C-Y Yang et al 2017; Ji et al 2019, incorporated by reference herein).

Gene ontology (GO) analyses integrating DEGs at 4 and 12 weeks showed that the biological processes, cell components and molecular functions relevant for cartilage development, extracellular matrix (ECM), ossification and hypertrophy are enriched in OA (data not shown), consistent with previous reports. Category network (cnet) analyses further confirm these observations and highlight the contribution of networks relevant to ECM assembly and signaling to OA (FIG. 3F).

### Example 4—The Progression of Osteoarthritis after DMM Surgery is Accompanied by Time-Dependent Methylation Patterns in Articular Cartilage

OA chondrocytes experience phenotypic and functional alterations that are in part related with changes in DNA methylation including changes in 5 hmC following DMM and an attempt to repair tissue damage (Ripmeester Ellen G-J PMID: 29616218; Singh et al 2018; Reynard et al; Shen J et al 2017, incorporated by reference herein). To evaluate if the structural and transcriptomic changes associated with DMM surgeries are also associated with changes in DNA methylation, we next conducted Reduced Representation Oxidative Bisulfite Sequencing (RRoxBS) analyses in DNA from cartilage samples retrieved at 4 and 12 weeks after DMM to assess changes in 5 mC (5-methylcytosine) and 5 hmC (5-hydroxymethyl-cytosine).

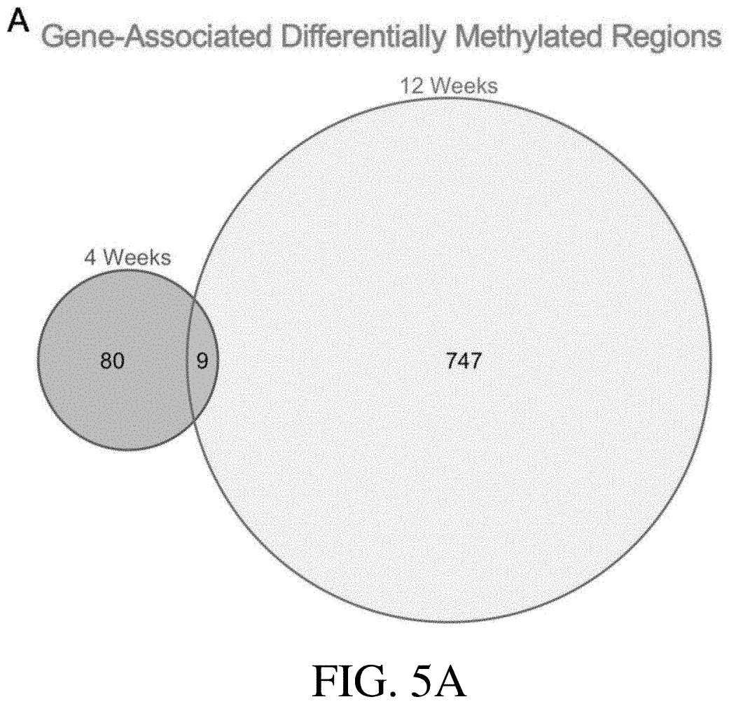

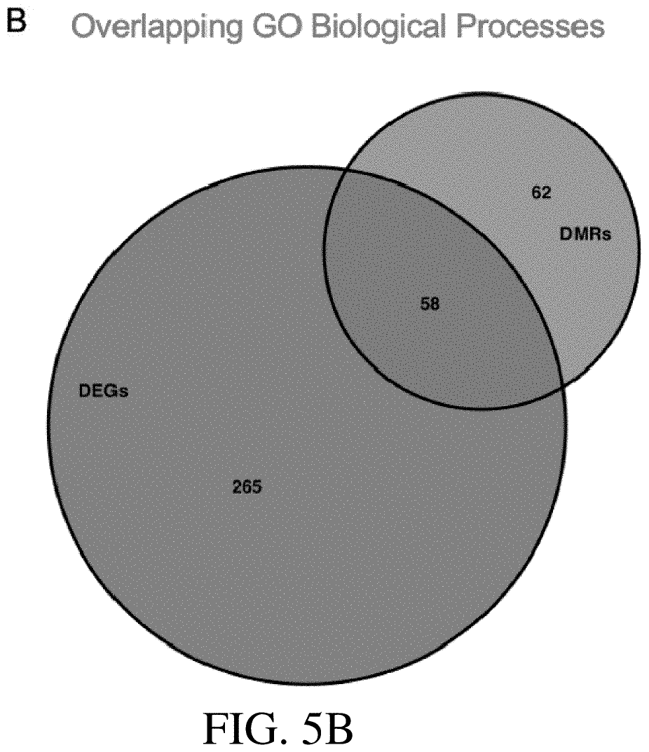

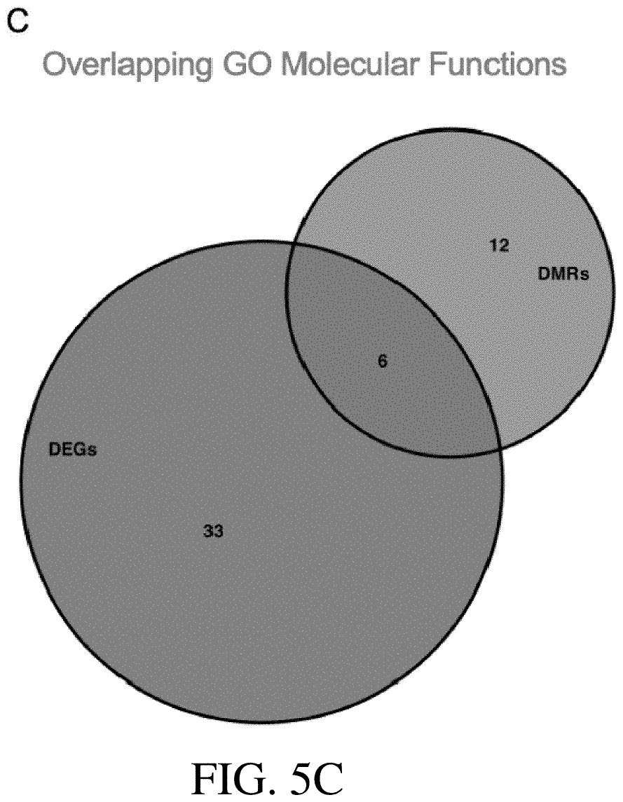

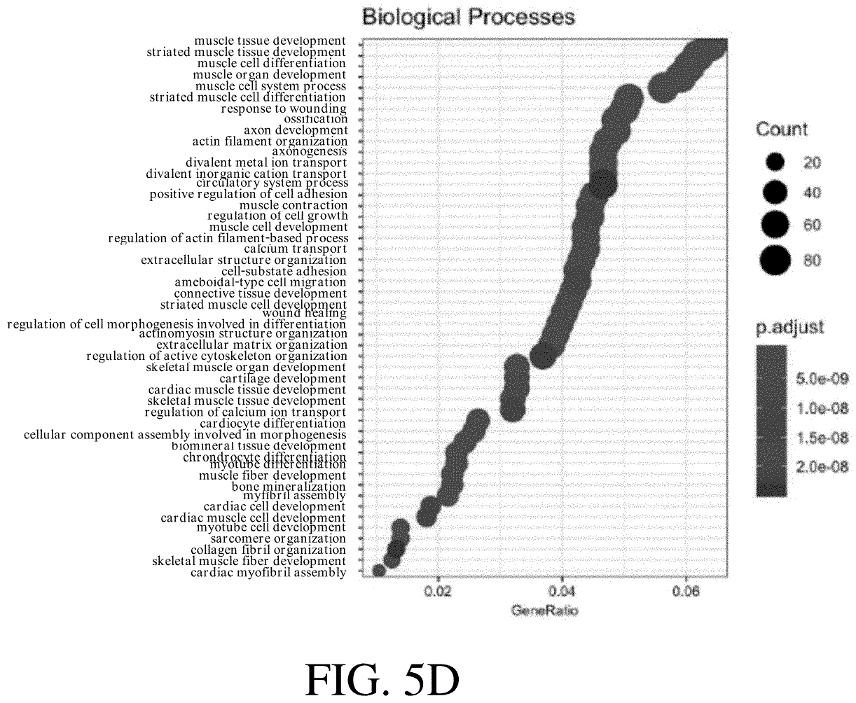

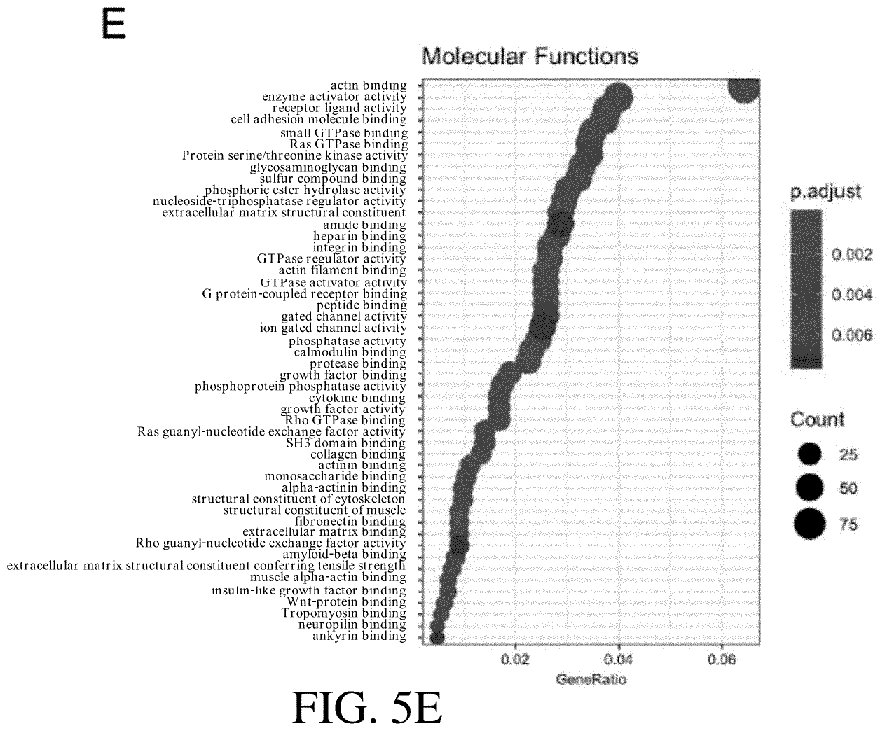

Comparisons between control and DMM-operated samples at 4 and 12 weeks after DNN uncovered significant differences in hyper- and hypo-methylation at both timepoints (data not shown). Using at least a 25% methylation difference and q-value <0.05 between DMM and control samples, we identified 842 differentially methylated 5 mCs and 318 5 hmCs at 4 weeks after DMM, and a dramatic increase in the number of differentially methylated cytosines (DMCs) at 12 weeks. This was particularly evident for 5 mCs, with 3614 differentially methylated 5 mCs and 480 5 hmCs (data not shown). Next, we used true methyl data (5 mC) to identify differentially methylated regions (DMR). We defined DMR as a genomic region with at least 3 CpGs within 100 bp, where at least 1 CpG is significantly differentially methylated (25% methylation difference and a q value <0.01) and the region has an overall average differential methylation of at least 20% across all the CpGs. We identified 89 DMRs associated with 90 unique gene symbols at 4 weeks, and 756 DMRs with 489 unique gene symbols associated with them at 12 weeks, with 9 DMRs common to 4 and 12 weeks (FIG. 5A).

Functional analyses using the 4 and 12 week RRoxBS data identified molecular functions (FIG. 5B) and biological processes (data not shown) enriched in our dataset, including functions relevant to ECM constituents, enzymatic binding and activity, or growth factor and cytokine binding. Integrative analyses of our RNAseq and RRoxBS datasets led to the identification of genes that are differentially methylated and differentially expressed at 4- and 12-weeks post-surgery (FIG. 5C), and functional integration of DEGs and DMRs at 4 and 12 weeks in GO categories revealed unique and overlapping biological processes enriched in OA cartilage after DMM surgery, with 33 biological processes unique to DEGs, 12 biological process unique to DMRs, and 6 biological processes common to both time-points (FIG. 5D and data not shown). Together, our transcriptomic and epigenomic analyses confirmed the changes in gene expression and DNA methylation reported using human samples and murine tissues and further suggest that the progression of OA is accompanied by time-dependent changes in the articular cartilage transcriptome and DNA methylome.

The time-dependent changes detected using bulk articular cartilage samples may be affected by the loss of cartilage cells due to the severe structural changes observed in established and late-stage OA disease, where most of the superficial zone chondrocytes are lost. To minimize the impact of cartilage loss in our downstream analyses, and to identify changes that may impact the early stages of the disease, we next focused primarily in the 4-week time point in subsequent analyses and comparisons.

### Example 5—The Increased Lrrc15 Expression in Early OA Cartilage is Associated with Decreased DNA Methylation of the LRRC15 Gene

To evaluate whether results obtained in the DMM model could be informative to address clinically-relevant changes in gene expression and DNA methylation, we next performed bioinformatics integration of our RNAseq and RRoxBS data with human OA RNAseq or DNA methylation datasets using HuGENet. Our analyses revealed notable parallels between the results obtained using the DMM model and human OA disease, but also highlighted differences that are driven by the type of tissues and platforms selected for the analyses (data not shown).

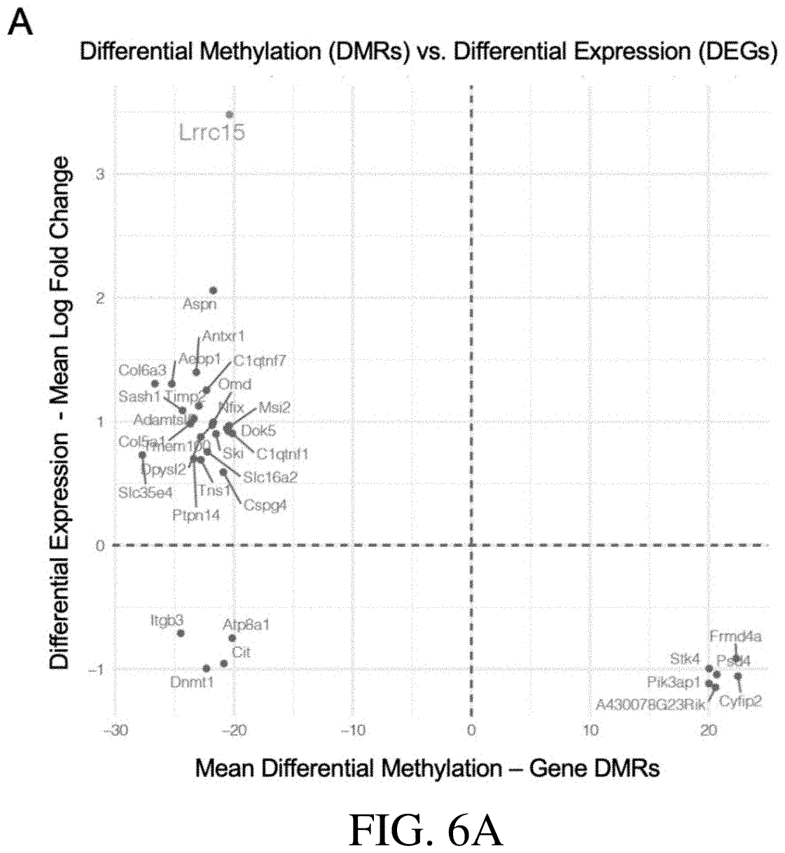

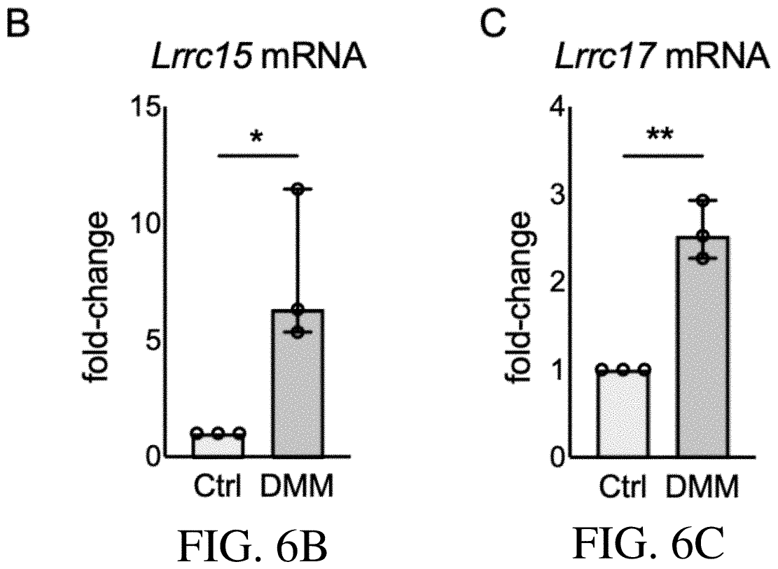

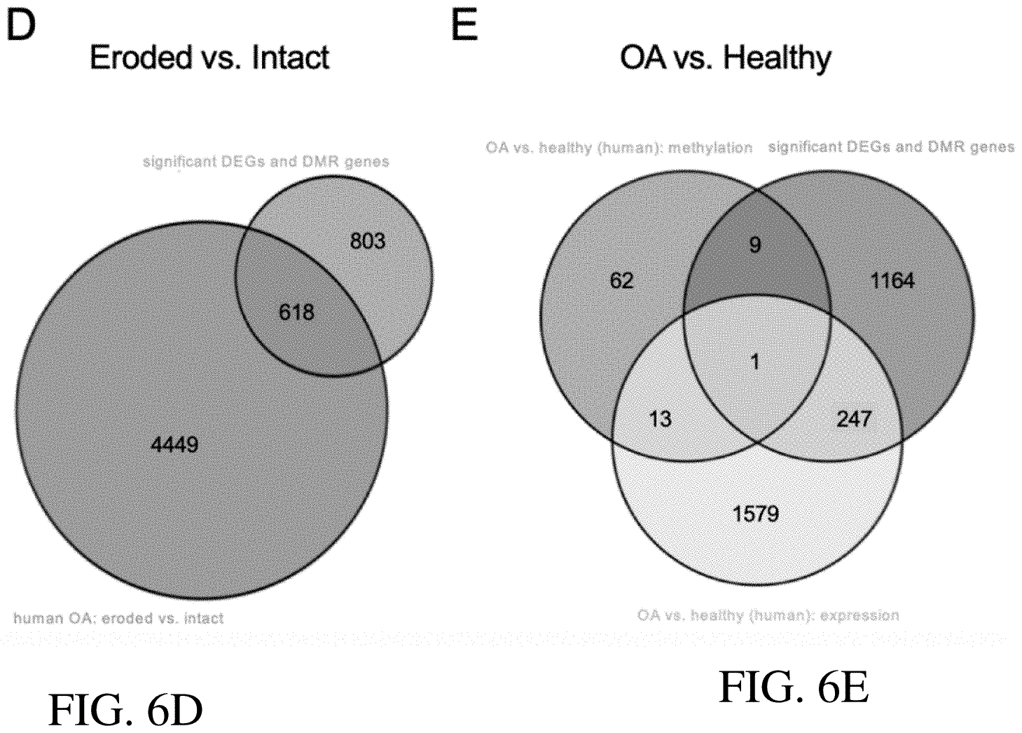

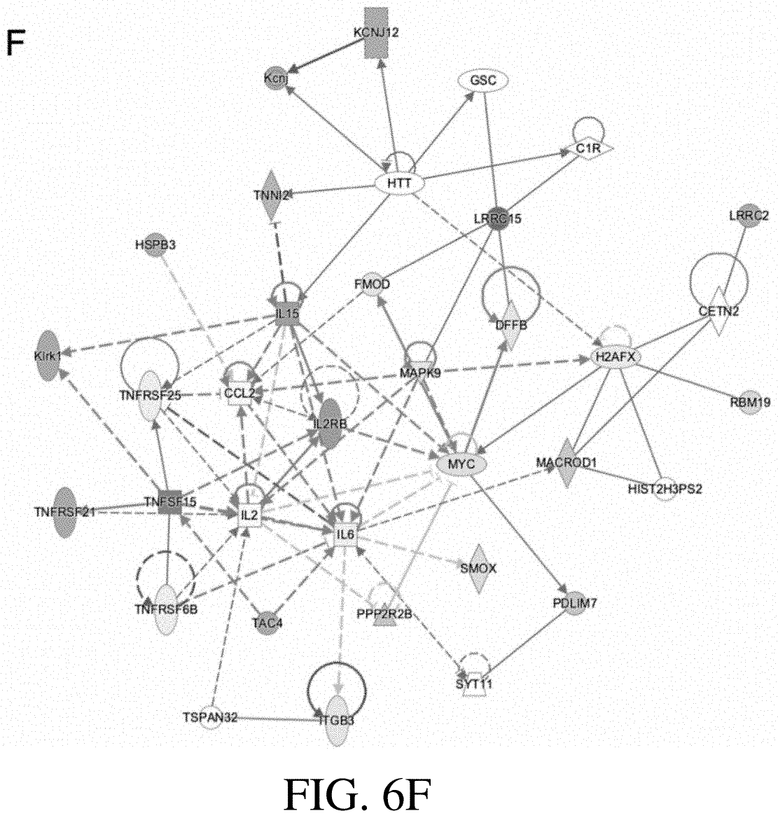

Next, we performed correlative analyses using our RNAseq and RRoxBS data, which revealed genes with changes in gene expression correlated with changes in DNA (5 mC) methylation (FIG. 6A). The Leucine Rich Repeat Containing 15 (LRRC15) gene emerged as the gene displaying the strongest inverse correlation between hypomethylation (−27.0067) and increased gene expression (3.5-fold) inversely correlated with methylation in early OA cartilage. We confirmed that LRRC15 expression was increased in early (4 week) cartilage samples after DMM by RTqPCR analyses (FIG. 6B), which also showed increased Lrrc17 mRNA (FIG. 6C) but without changes in 5 mC methylation also in agreement with our RNAseq data. The increased expression of LRRC15 in OA cartilage after DMM was consistent with previous reports in human OA cartilage as identified by the integration of our data and human datasets (see, also Chen Yi-Jen et al 2017, 2018 and Karlson C et al 2009; Ji et al 2019, incorporated by reference), suggesting its potential contribution to OA disease. These comparisons highlighted notable disease stage- and platform-dependent differences within human datasets. Comparisons with HuGENet identified 28 overlapping genes (out of 168 OA-associated genes), including 9 genes with gene associated-DMRs (Havcr2, Ncor2, Aspn, Tnfrsf11b, Smad3, Tcf711, Lrp5, Fos, and Pepd). We further separated the published datasets onto two comparator groups: eroded vs. non-eroded OA cartilage, with 618 overlapping genes (FIG. 6D), and healthy vs. OA cartilage with 248 DEGs and 10 DMR associated genes overlapping, and Runx1 as the gene at the intersect between methylation and expression in published human datasets and our mouse data (FIG. 6E). Bioinformatics analyses showed that LRRC15 belongs to the collagen binding network enriched in OA (data not shown), and analyses of the 4-week datasets shows the interaction of LRRC15 with other genes with differential expression and changes in DNA methylation in OA, as shown in the Cnet plot of molecular functions network (FIG. 6F).

We mined our datasets to evaluate additional interactions of LRRC15 with differentially expressed or methylated genes at 4 weeks after DMM. The cnet plot of molecular functions shown in FIG. 6F represents the integration and interaction of LRRC15 in a network that includes factors that contribute to signaling, apoptosis, or inflammation. Thus, our integrative analyses confirmed that the increased expression of LRRC15 is conserved in human and mouse OA cartilage, and suggest a potential functional involvement of LRRC15 in OA disease.

### Example 6—LRRC15 Immunostaining in Human and Murine Cartilage Samples

Next, we evaluated the presence of LRRC15 protein in human and mouse OA cartilage samples. LRRC15 protein was present in human cartilage retrieved from patients undergoing total knee replacement for OA (N=5). A Safranin 0-stained tissue showed relatively intact structure, retaining superficial cartilage (data not shown). Adjacent serial sections were used for LRRC15 immunostaining, which showed LRRC15 protein distributed throughout all the cartilage zones. LRRC15 immunostaining was observed in all human OA cartilage samples, independent of the severity of the structural damage Similarly, we selected control and DMM-operated mouse tissues at 4 weeks after surgery for LRRC15 immunostaining We stained control and DMM-operated tissues with Safranin 0 and Fast green, and we incubated adjacent sections with anti-LRRC15 antibodies. We detected minimal presence of LRRC15 immunostaining in the control tissues relative to background signal. In agreement with our RNA-seq and qPCR data, the DMM-operated tissues showed increased LRRC15 signal relative to control samples. The increased LRRC15 positive immunostaining was particularly prominent in the deep/calcified cartilage zones in DMM-operated tissues, but also observed in superficial chondrocytes. LRRC15 immunostaining was also very prominent in areas of osteophyte formation in DMM-operated limbs, and in the hypertrophic zones in the postnatal growth plates in control (not shown) and DMM samples.

LRRC15LRRC15LRRC15Together, these results confirmed the presence of LRRC15 protein in human and murine articular cartilage and further suggested that increased LRRC15 may contribute to disease progression and to changes in OA chondrocyte phenotype and responses.

### Example 7—LRRC15 Expression is Induced by Inflammatory Cytokines and DNA Demethylation in Articular Chondrocytes In Vitro

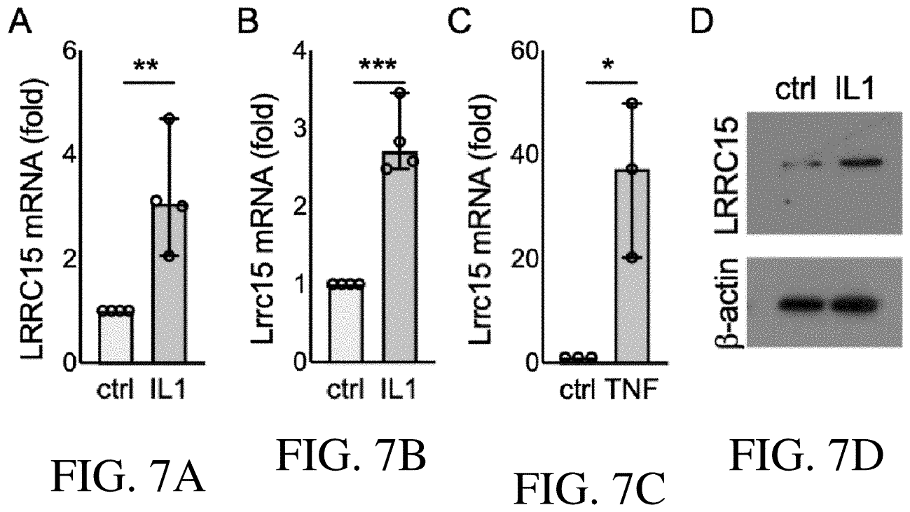

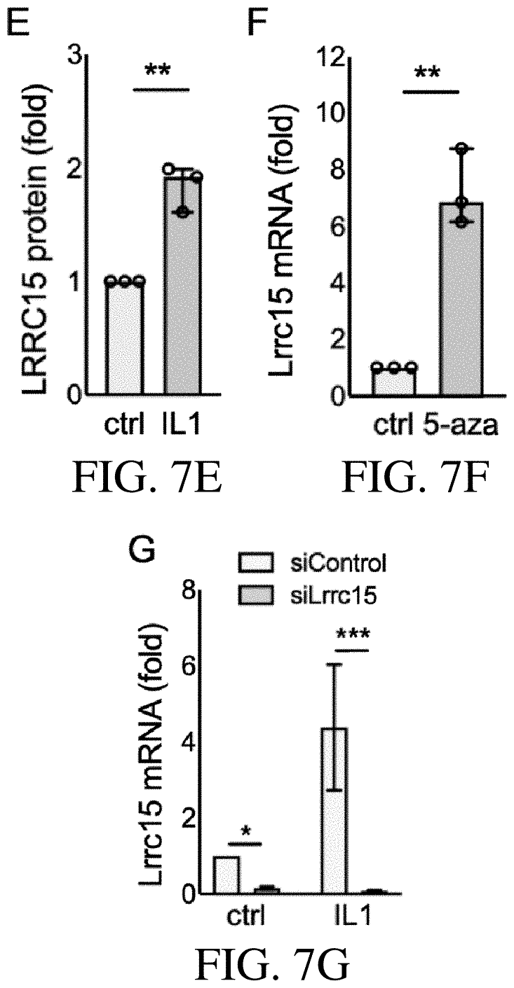

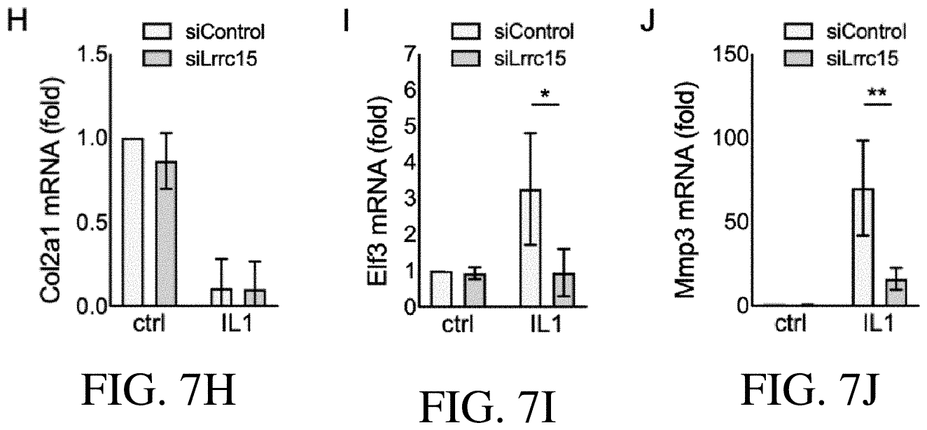

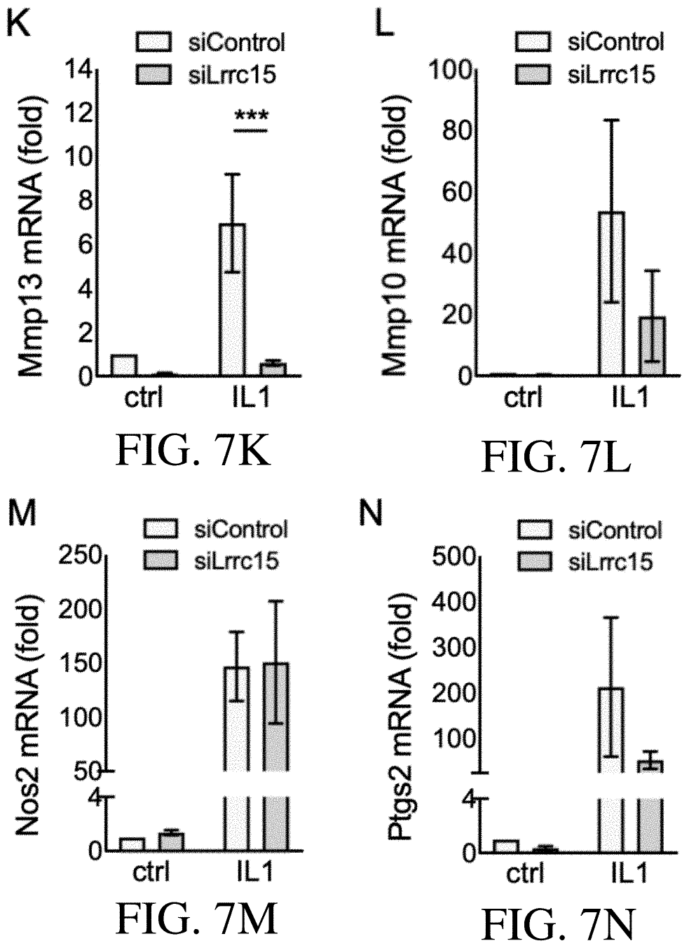

We next investigated changes in LRRC15 expression using human and murine chondrocytes treated with inflammatory cytokines in vitro, to mimic OA-like changes (Loughlin et al 2014; 5; Goldring M B et al 2012; Olivotto E et al 2015; Hashimoto et al 2009, incorporated herein by reference). Consistent with studies showing cytokine-induced expression in other cell types (Wang Y et al 2018 PMID: 29523191; Satao et al, incorporated herein by reference), IL-113 treatment induced increased LRRC15 mRNA (FIG. 7A) and protein (data not shown) in cell lysates from human primary chondrocytes. We next used murine primary chondrocytes and confirmed that IL-1β (FIG. 7B) and TNFα (FIG. 7C) induced LRRC15 mRNA, and that IL-1β treatment also lead to increase LRRC15 protein (FIGS. 7D and 7E). Previous studies showed that the long-term stimulation of articular chondrocytes with cytokines leads to long-lasting changes in gene expression (Hashimoto 2009, incorporated by reference).

We also found that long-term stimulation of mouse chondrocytes with IL-1β lead to a sustained increased in LRRC15 mRNA expression even after cytokine withdrawal and cell passage (data not shown). This observation, together with our RNAseq and RRoxBS data in cartilage after DMM, suggested that changes in DNA methylation may have a functional impact in LRRC15 transcription. To test this, we treated murine primary chondrocytes with the DNA methyl transferase inhibitor, 5-Aza-2′-deoxycytidine (5-aza), alone (data not shown) or combined with the histone deacetylase inhibitor trichostatin (TS) (FIG. 7E), as previously shown (Hashimoto 2009, incorporated by reference). Treatment with 5-aza and TS lead to an early (72 hours) and sustained (1 week) increase in LRRC15 expression in murine chondrocytes (FIG. 7E) accompanied by increased Mmp13 mRNA (FIG. 7F), which was used as positive control for 5-aza+TS treatment (Hashimoto 2009). Together, these results suggest that the LRRC15 gene transcription in chondrocytes is at least in part driven by DNA de-methylation.

### Example 8—LRRC15 Contributes to the Il-1β-Induced Gene Expression in Articular Chondrocytes In Vitro

Finally, to understand the functional impact of LRRC15 in articular chondrocytes, we evaluated the impact of LRRC15 knockdown on the IL-1β-driven responses in articular chondrocytes. To do this end, we first tested the knockdown (KD) efficacy of 3 different custom-designed siRNA oligos against mouse LRRC15 (siLRRC15) relative to scramble non-targeting controls (siControl). We selected siLRRC15 oligo 1 (see Table 1) because it significantly reduced LRRC15 mRNA at 72 hours after transfection without impacting Lrrc17 mRNA, or the expression of cartilage-specific genes, Col2a1 and Sox9. The other two oligos tested showed similar LRRC15 knockdown efficacy but less specificity (data not shown).

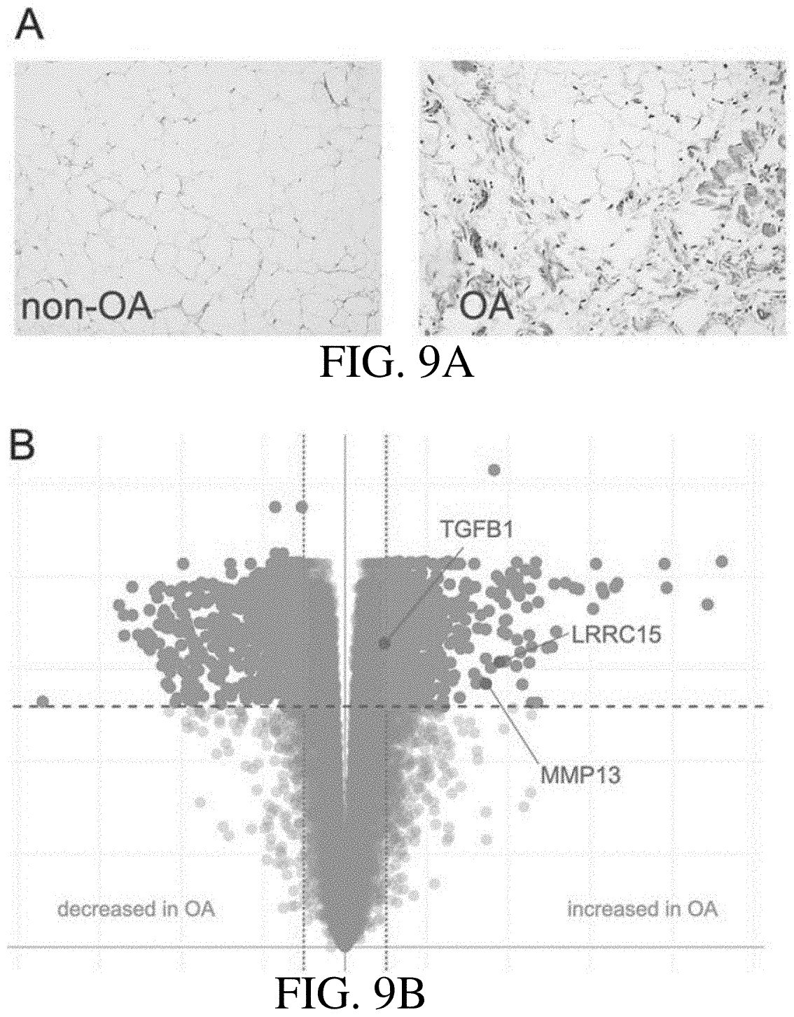

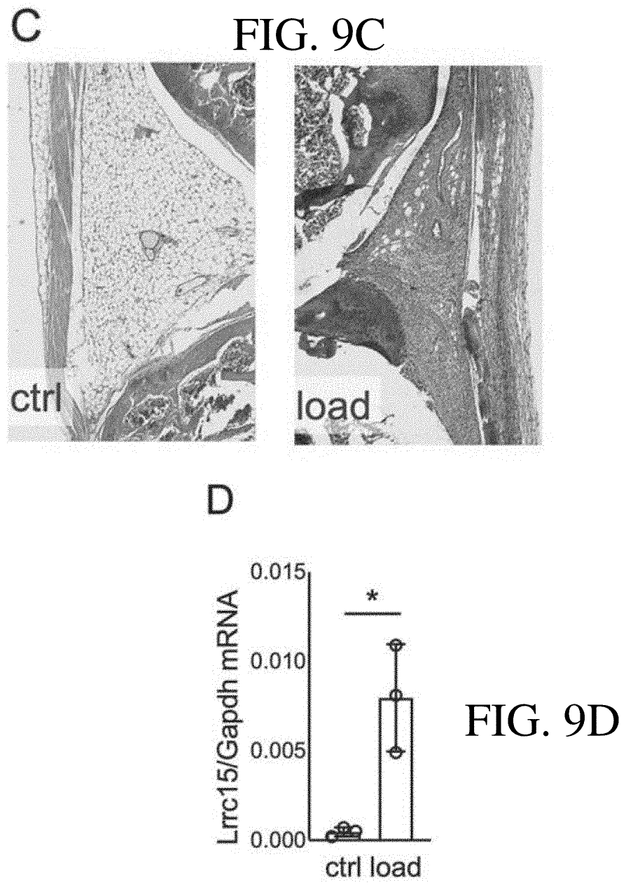

Next, we transfected murine primary chondrocytes with siControl or siLRRC15 oligos and we treated control (siControl) or LRRC15 KD (siLRRC15) murine primary chondrocytes with 1 ng/ml of IL-113 for 72 hours, and we evaluated the expression of cartilage-specific and OA-relevant genes. As shown in FIG. 7G, siLRRC15 cells displayed reduced LRRC15 mRNA at baseline and after IL-1β treatment. The IL-1β-driven repression of Acan and Col2a1 was not significantly different between siControl and siLRRC15 cells (FIG. 7B). However, the IL-1β-induced expression of Elf3 (FIG. 7I), Mmp13 (FIG. 7K), and Ptgs2 (FIG. 7N) was significantly reduced in siLRRC15 cells. The levels of other MMPs involved in cartilage catabolism, like Mmp3 (FIG. 9E) and Mmp10 (FIG. 7L) showed a non-significant reduction in IL-1β-induced expression in siLRRC15 cells, whereas the IL-113-driven expression of Nos2 remained unchanged after LRRC15 KD (FIG. 7M). Together, our results suggest that LRRC15 contributes in a gene-specific manner to the IL-1β-driven expression of genes involved in matrix remodeling and cartilage catabolism in OA.

Our integrative analyses and the comparison with human datasets led to the identification of epigenomic signatures that overlap with changes in gene expression, with enrichment of pathways relevant to cartilage development. We also identified LRRC15 as a gene with differential expression and 5 mC hypomethylation in the early disease stages, and with contribution to the IL-1β-induced responses of chondrocytes in vitro.

Our RNA-seq data is enriched in genes and functional pathways relevant to cartilage development, hypertrophy, and ossification. This is consistent with previous studies using human and murine cartilage samples, and further reinforces the notion that OA chondrocytes undergo a phenotypic shift and recapitulate developmental steps in an attempt to repair tissue damage. Interestingly, while the enrichment in cell-cell and cell-matrix interaction, hypertrophy, ossification, and ECM assembly pathways are constant, the specific genes up and down-regulated differ between the 4- and 12-week time-points. This could be a consequence of gene-specific transcriptional kinetics and temporal engagement of different transcriptional networks, but it also suggests that whole-tissue transcriptomic analyses can be partly reflecting loss of cartilage structure in more advanced OA disease and therefore loss of specific cellular subsets that are responding to different stimuli and expressing a different array of OA-related genes. More importantly, these time-specific changes highlight the need for developing targeted approaches that take into account disease stage-specific transcriptional changes.

Our RRoxBS data agrees with these studies, showing profound changes in 5 mC and 5 hmC patterns accompanying structural and transcriptional changes during the progression of OA after DMM. Integrating RNA-seq and 5 mC data we found that changes in DNA methylation are associated with an enrichment of developmental pathways in OA chondrocytes. We observed more pronounced 5 mC changes relative to the changes observed in 5 hmC in our analyses which may be due to the different platforms used to assay and analyze DNA methylation patterns. RRoxBS selects for GC-rich genomic regions and covers the majority of gene promoters and CpG islands, but provides limited coverage of CpG shores and other relevant intergenic regions that accumulate 5 hmC during the progression of OA. These differences notwithstanding, our data provides further evidence of the impact of changes in 5 mC to OA, and highlights the need for evaluating 5 mC/5 hmC homeostasis to dissect their relative contribution to the disease.

Integration of our RNA-seq and RRoxBS datasets allowed us to identify changes in gene expression associated with changes in DNA methylation patterns following DMM surgery, and additional bioinformatics comparisons with human data enabled us to uncover clinically relevant targets and changes in early disease stage. These integrative analyses highlighted LRRC15 as one of the genes with increased expression and significant 5 mC hypomethylation in early OA cartilage.

We found increased LRRC15 mRNA and protein levels upon cytokine stimulation of human and murine cells, and increased LRRC15 immunostaining in OA cartilage. We also found a very prominent LRRC15 positive immunostaining in postnatal growth plates and the developing osteophytes, and our bioinformatics analyses showed that LRRC15 participates in collagen binding networks and inflammatory signaling. LRRC15 knockdown lead to reduced IL-1β-driven expression of a number of Mmp13 and Elf3 in chondrocytes, whereas other known direct canonical NF-kB targets like Nos2 and Ptgs2 were not affected by the LRRC15 knockdown. Thus, it is conceivable that LRRC15 drives gene expression in a cell and gene-specific context, likely via concerted modulation of canonical NF-kB and other signaling pathways. Taken together, our data suggests that increased LRRC15 levels in early OA represents an early event in the chondrocyte activation characteristic of OA which, in an attempt to repair tissue damage recapitulating developmental processes, may in turn contribute to disease progression and to permanent changes in OA chondrocyte phenotype and responses.

The integration of our datasets with human orthologs using HuGENet confirmed the utility of the DMM model as a preclinical exploratory tool and identified conserved OA-related changes in gene expression and DNA methylation. In summary, these data provide new insights about the contribution of 5 mC changes to cartilage damage in OA, and highlights LRRC15 as a gene with potential contribution to OA disease.

### Example 9— Additional Preliminary Experiments

In preliminary experiments, we also detected increased LRRC15 mRNA in human infrapatellar fat pad from OA patients, and in purified primary fibroblast-like synoviocytes treated with TGFβ1. Using primary human and murine chondrocytes, we showed that DNA demethylation leads to increased LRRC15 mRNA expression in vitro. Treatment with cytokines relevant to OA disease (IL-1β and TNFα) also leads to increased LRRC15 mRNA and protein in chondrocytes. Using murine primary chondrocytes, we knocked down LRRC15 and found that it contributes to the IL-113-driven expression of catabolic genes relevant to OA disease, including Mmp13 and Ptgs2.

Additional preliminary data (not shown) supports that (1) LRRC15 knockdown leads to decreased expression of IL1-induced catabolic genes, (2) TGFβ1 treatment leads to increase expression of LRRC15, and (3) LRRC15 mRNA is increased in human and mouse OA infrapatellar fat pads, suggesting that it may contribute to the overall knee joint damage in OA.

### Example 10—Defining the Mechanism/s of Action of Lrrc15 in OA Relevant Tissues

Short-term, we better define the mechanisms of action of LRRC15 in OA relevant tissues (e.g. cartilage, adipose tissue, synovium, meniscus) in vitro and in vivo, to begin to understand its functional impact on joint homeostasis and OA. Initial experiments evaluate the impact of deficient LRRC15 expression (and activity) to OA disease using LRRC15 knockout/conditional knockout mice undergoing experimental (surgical and non-surgical) induction of OA, followed by evaluation of structural and behavioral (e.g. pain) changes and in vitro systems.

Long term, epigenome/genome editing is implemented to address how the modulation of LRRC15 expression impacts joint homeostasis and the progression of osteoarthritis. Follow-up experiments involve modification of LRRC15 expression using gene silencing by delivery of siRNA targeting LRRC15 RNA.

We also evaluate the mechanism/s of action of LRRC15 in homeostasis and pathology in chondrocytes and other relevant cells in vitro and in vivo.

### Example 11— Intraarticular Anti-Lrrc15 Antibody Delivery to Treat OA-Associated Fibrosis, Progression and Symptoms in Patients with Early OA

In one embodiment, modification of LRRC15 gene expression and/or activity is expected to prevent or slow down the progression of osteoarthritis. In one embodiment, modification of LRRC15 expression is achieved via intra-articular delivery of LRRC15 siRNA oligonucleotides.

In another embodiment, modification of LRRC15 activity is achieved by local delivery, i.e., intra-articular injection, of anti-LRRC15 antibodies as shown using conventional or tissue-specific knockout mice. Antibodies that target LRRC15 activity permit the testing of its efficacy as a therapeutic target.

Intra-articular drug delivery is commonly used in patients with osteoarthritis (OA), and patients with OA often receive intra-articular injections of steroids or platelet-rich-plasma to treat symptoms. Intra-articular injections are safe, ensure local delivery of the treatment, and avoid potential side effects associated with systemic delivery.

Our previous results showed increased LRRC15 mRNA and protein in patients with knee OA, and that LRRC15 blockade (in vitro, using siRNA) lead to reduced expression of genes involved in inflammation and cartilage degradation. We also found an association between increased knee fibrosis and increased LRRC15 levels. Building on these data, a randomized, double-blind, placebo-controlled study is conducted as follows: The safety and tolerability of up to 5 different anti-LRRC15 doses administered intra-articularly (starting dose 100 mg, maximum dose 500 mg) is observed by administering as an ascending single dose. Participants receive a single intra-articular injection of anti-LRRC15 (ABBV 085) from 100 to 500 mg by intra-articular injection. A control is administered placebo or inert vehicle by intra-articular injection.

Thereafter a randomized trial is conducted in which we assess structural changes (fibrosis and cartilage degradation), knee stiffness (range-of-motion) and reduction in pain at 1 year, in response to a single intra-articular injection of a selected dose of anti-LRRC15 compared to placebo and conventional therapy (acetaminophen). Participants are randomized to receive a single intra-articular injection of anti-LRRC15 (dose selected in part #1), placebo, or acetaminophen tablets orally. The anti-LRRC15 (e.g. ABBV 085) is delivered via intra-articular injection). The placebo is administered to a first control patient via intra-articular injection. The active agent acetaminophen is delivered orally.

### Example 12: Lrrc15 as a Biomarker to Identify OA Patients with Fibrosis

The above examples demonstrate increased LRRC15 in fibrotic joint tissues, and changes in LRRC15 protein levels in synovial tissues from knee OA patients and patients undergoing ACL reconstruction surgery who had evidence of high inflammation and fibrosis histologically.

An antibody to LRRC15, such as ABBV 085, is employed as a predictive tool, to identify knee OA patient subtypes characterized by the early presence of fibrosis. These patients may be at high risk of progressing towards late-stage disease.

In one embodiment, a sample of the patients joint tissue or synovial fluid or other joint tissue is obtained. ABBV085 to which a fluorescent label is attached is contacted with the sample in vitro and levels of LRRC15 are measured in the sample. The sample is compared with a control, which indicates normal levels of LRRC15 in the tissue of healthy, non-arthritic subjects. An increase in detectable LRRC15 bound to the labeled ABBV 085 over the control is indicative of a diagnosis of early stage, or progressing OA. Anti-LRRC15 blockade may be used to prevent or slow down inflammation, fibrosis, and structural progression.

Each and every patent, patent application, and publication, including websites cited throughout specification are incorporated herein by reference. Similarly, the SEQ ID NOs which are referenced herein, and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### REFERENCES

1. Singh P, et al. Phenotypic instability of chondrocytes in osteoarthritis: on a path to hypertrophy. Ann N Y Acad Sci. 2019 April; 1442(1):17-34. doi: 10.1111/nyas.13930. Epub 2018 Jul. 15. Review. PMID: 30008181
2. Steinberg J, et al, Integrative epigenomics, transcriptomics and proteomics of patient chondrocytes reveal genes and pathways involved in osteoarthritis. Sci Rep. 2017 Aug. 21; 7(1):8935PMID:28827734
3. Culley K L, et al., Mouse models of osteoarthritis: surgical model of posttraumatic osteoarthritis induced by destabilization of the medial meniscus. Methods Mol Biol. 2015; 1226:143-73. doi: 10.1007/978-1-4939-1619-1_12. PubMed PMID: 25331049. https://www.ncbi.nlm nih.gov/pubmed/25331049
4. Chen Y J, et al., Systematic Analysis of Transcriptomic Profile of Chondrocytes in Osteoarthritic Knee Using Next-Generation Sequencing and Bioinformatics. J Clin Med. 2018 Dec. 10; 7(12) (PMID-30544699).
5. Otero M, et al., Human chondrocyte cultures as models of cartilage-specific gene regulation. Methods Mol Biol. 2012; 806:301-36. doi: 10.1007/978-1-61779-367-7_21. PubMed PMID: 22057461.
6. Otero M, et al., E74-like factor 3 (ELF3) impacts on matrix metalloproteinase 13 (MMP13) transcriptional control in articular chondrocytes under proinflammatory stress. J Biol Chem. 2012 Jan. 27; 287(5):3559-72. doi: 10.1074/jbc.M111.265744. Epub 2011 December PubMed PMID: 22158614; PMCID: PMC3271009.
7. Wondimu E B, et al. Elf3 Contributes to Cartilage Degradation in vivo in a Surgical Model of Post-Traumatic Osteoarthritis. Sci Rep. 2018 Apr. 24; 8(1):6438. doi: 10.1038/s41598-018-24695-3. PMID: 29691435
8. Chen L Y, et al. Modulation of matrix metabolism by ATP-citrate lyase in articular chondrocytes. J Biol Chem. 2018; 293(31):12259-12270. doi:10.1074/jbc.RA118.002261
9. Chen Y J, et al. Deduction of Novel Genes Potentially Involved in Osteoblasts of Rheumatoid Arthritis Using Next-Generation Sequencing and Bioinformatic Approaches. Int J Mol Sci. 2017; 18(11):2396. Published 2017 Nov. 11. doi:10.3390/ijms18112396
10. Cuellar T L, et al. Systematic evaluation of antibody-mediated siRNA delivery using an industrial platform of THIOMAB-siRNA conjugates. Nucleic Acids Res. 2015; 43(2):1189-1203. doi:10.1093/nar/gku1362
11. Dolan J, et al. The extracellular leucine-rich repeat superfamily; a comparative survey and analysis of evolutionary relationships and expression patterns [published correction appears in BMC Genomics. 2009; 10:230]. BMC Genomics. 2007; 8:320. Published 2007 Sep. 14. doi:10.1186/1471-2164-8-320
12. Dunn S L, et al. Gene expression changes in damaged osteoarthritic cartilage identify a signature of non-chondrogenic and mechanical responses. Osteoarthritis Cartilage. 2016; 24(8):1431-1440. doi:10.1016/j.joca.2016.03.007
13. Fernandez-Tajes J, et al. Genome-wide DNA methylation analysis of articular chondrocytes reveals a cluster of osteoarthritic patients. Ann Rheum Dis. 2014; 73(4):668-677. doi:10.1136/annrheumdis-2012-202783
14. Gayatri S, et al. Using oriented peptide array libraries to evaluate methylarginine-specific antibodies and arginine methyltransferase substrate motifs. Sci Rep. 2016 June; 6:28718. doi:10.1038/5rep287189.
15. Gennaro, A. R., Remington: The Science and Practice of Pharmacy, (Lippincott, Williams and Wilkins);
16. Goldring M B, Marcu K B. Cartilage homeostasis in health and rheumatic diseases. Arthritis Res Ther. 2009; 11(3):224. doi:10.1186/ar2592
17. Goldring M. B., Marcu K. B. (2012) Epigenomic and microRNA-mediated regulation in cartilage development, homeostasis, and osteoarthritis. Trends Mol Med 18: 109-118
18. Hashimoto K., et al. (2009) DNA demethylation at specific CpG sites in the IL1B promoter in response to inflammatory cytokines in human articular chondrocytes. Arthritis Rheum 60: 3303-3313
19. Heerboth et al. Use of Epigenetic Drugs in Disease: An Overview. Genetics & Epigenetics 2014:6 9-19 doi:10.4137/GEG.S12270;
20. Jeffries M A et al. Genome-wide DNA methylation study identifies significant epigenomic changes in osteoarthritic cartilage. Arthritis Rheumatol. 2014; 66(10):2804-2815. doi:10.1002/art.38762
21. Jeffries, M. A. et al. (2016). Genome-Wide DNA Methylation Study Identifies Significant Epigenomic Changes in Osteoarthritic Subchondral Bone and Similarity to Overlying Cartilage. Arthritis & rheumatology (Hoboken, N.J.), 68(6), 1403-1414. https://doi.org/10.1002/art.39555
22. Ji Q, et al. Single-cell RNA-seq analysis reveals the progression of human osteoarthritis. Ann Rheum Dis. 2019; 78(1):100-110. doi:10.1136/annrheumdis-2017-212863
23. Dehne T, et al. Chondrogenic differentiation potential of osteoarthritic chondrocytes and their possible use in matrix-associated autologous chondrocyte transplantation. Arthritis Res Ther. 2009; 11(5):R133. doi:10.1186/ar2800
24. Karlsson C, et al. Genome-wide expression profiling reveals new candidate genes associated with osteoarthritis. Osteoarthritis Cartilage. 2010; 18(4):581-592. doi:10.1016/j.joca.2009.12.002
25. Kibbe, A. H., & American Pharmaceutical Association. (2000). Handbook of pharmaceutical excipients. Washington, D.C: American Pharmaceutical Association.
26. Lan Yi, et al., Selected drugs that inhibit DNA methylation can preferentially kill p53 deficient cells. 2014 October, Oncotarget. 5(19): 8924-8936
27. Li H, et al. Whole-transcriptome sequencing of knee joint cartilage from osteoarthritis patients. Bone Joint Res. 2019; 8(7):290-303. Published 2019 Aug. 2. doi:10.1302/2046-3758.87.BJR-2018-0297.R1
28. Lieberman, H. A., et al. (1998). Pharmaceutical dosage forms: Disperse systems: vol. 3. New York: Marcel Dekker.
29. Liu Y, et al. Chromatin accessibility landscape of articular knee cartilage reveals aberrant enhancer regulation in osteoarthritis. Sci Rep. 2018; 8(1):15499. Published 2018 Oct. 19. doi:10.1038/s41598-018-33779-z
30. Loeser R F, et al. Disease progression and phasic changes in gene expression in a mouse model of osteoarthritis. PLoS One. 2013; 8(1):e54633. doi:10.1371/journal.pone.0054633
31. Loughlin J, Reynard L N. Osteoarthritis: Epigenetics of articular cartilage in knee and hip O A. Nat Rev Rheumatol. 2015; 11(1):6-7. doi:10.1038/nrrheum.2014.189
32. Marcu K B, et al., N F-kappaB signaling: multiple angles to target O A. Curr Drug Targets. 2010; 11(5):599-613. doi:10.2174/138945010791011938
33. National Academies of Sciences, Engineering, and Medicine. 2017. Human Genome Editing: Science, Ethics, and Governance. Washington, D C: The National Academies Press. https://doi.org/10.17226/24623
34. Olivotto E, et al. Pathophysiology of osteoarthritis: canonical NF-κB/IKKβ-dependent and kinase-independent effects of IKKα in cartilage degradation and chondrocyte differentiation. RMD Open. 2015; 1(Suppl 1):e000061. Published 2015 Aug. 15. doi:10.1136/rmdopen-2015-000061
35. Remington, J. P., & Gennaro, A. R. (2000). Remington: The science and practice of pharmacy. Baltimore, Md.: Lippincott Williams & Wilkins.
36. Ren Y M, et al. Exploring the Key Genes and Pathways of Osteoarthritis in Knee Cartilage in a Rat Model Using Gene Expression Profiling. Yonsei Med J. 2018; 59(6):760-768. doi:10.3349/ymj.2018.59.6.760
37. Reynard L N. Analysis of genetics and DNA methylation in osteoarthritis: What have we learnt about the disease?. Semin Cell Dev Biol. 2017; 62:57-66. doi:10.1016/j.semcdb.2016.04.017
38. Reynolds P A, et al. Identification of a DNA-binding site and transcriptional target for the EWS-WT1(+KTS) oncoprotein. Genes Dev. 2003; 17(17):2094-2107. doi:10.1101/gad.1110703
39. “Ripmeester E G J, et al. Recent Insights into the Contribution of the Changing Hypertrophic Chondrocyte Phenotype in the Development and Progression of Osteoarthritis. Frontiers in Bioengineering and Biotechnology. 2018; 6:18. DOI: 10.3389/fbioe.2018.00018
40. Roman-Blas J A, Jimenez S A. NF-kappaB as a potential therapeutic target in osteoarthritis and rheumatoid arthritis. Osteoarthritis Cartilage. 2006; 14(9):839-848. doi:10.1016/j.joca.2006.04.008
41. Saklatvala J. Inflammatory signaling in cartilage: MAPK and NF-kappaB pathways in chondrocytes and the use of inhibitors for research into pathogenesis and therapy of osteoarthritis. Curr Drug Targets. 2007; 8(2):305-313. doi:10.2174/138945007779940115
42. Satoh K, et al. A novel member of the leucine-rich repeat superfamily induced in rat astrocytes by beta-amyloid. Biochem Biophys Res Commun. 2002; 290(2):756-762. doi:10.1006/bbrc.2001.6272
43. Sebastian A, et al. Comparative Transcriptomics Identifies Novel Genes and Pathways Involved in Post-Traumatic Osteoarthritis Development and Progression. Int J Mol Sci. 2018; 19(9):2657. Published 2018 Sep. 7. doi:10.3390/ijms19092657
44. Serrano-Sevilla, I. et al., Natural Polysaccharides for siRNA Delivery: Nanocarriers Based on Chitosan, Hyaluronic Acid, and Their Derivatives, Molecules 2019 July; 24(14): 2570 PMID: 31311176;
45. Shen J, et al. Inflammation and epigenetic regulation in osteoarthritis. Connect Tissue Res. 2017; 58(1):49-63. doi:10.1080/03008207.2016.1208655
46. Soul J, et al., SkeletalVis: an exploration and meta-analysis data portal of cross-species skeletal transcriptomics data. Bioinformatics. 2019; 35(13):2283-2290. doi:10.1093/bioinformatics/bty947
47. U.S. Pat. No. 10,195,209
48. US Patent Publication No. 20200149026
49. US Patent Publication No. US2013/0129668 (Firestein)
50. Wang Y, et al. LRRC15 promotes osteogenic differentiation of mesenchymal stem cells by modulating p65 cytoplasmic/nuclear translocation. Stem Cell Res Ther. 2018; 9(1):65. Published 2018 Mar. 9. doi:10.1186/s13287-018-0809-1
51. Yang C Y, et al. ADAMTS and ADAM metalloproteinases in osteoarthritis-looking beyond the ‘usual suspects’. Osteoarthritis Cartilage. 2017; 25(7):1000-1009. doi:10.1016/j.joca.2017.02.791

TABLE II

| SEQ ID NO: | |
|---|---|
| (containing | |
| free text) | Free text under <223> |
| 7 | <223> Synthetic polypeptide |
| 8 | <223> Synthetic polypeptide |
| 9 | <223> Synthetic polypeptide |
| 10 | <223> Synthetic polypeptide |
| 11 | <223> Synthetic polypeptide |
| 12 | <223> Synthetic polypeptide |
| 13 | <223> Synthetic polypeptide |
| 14 | <223> Synthetic polypeptide |
| 15 | <223> Synthetic polypeptide |
| 16 | <223> Synthetic polypeptide |
| 17 | <223> Synthetic polypeptide |
| 18 | <223> Synthetic polypeptide |
| 19 | <223> Synthetic polypeptide |
| 20 | <223> Synthetic polypeptide |
| 21 | <223> Synthetic polypeptide |
| 22 | <223> Synthetic polypeptide |
| 23 | <223> Synthetic polypeptide |
| 24 | <223> Synthetic polypeptide |
| 25 | <223> Synthetic polypeptide |
| 26 | <223> Synthetic polypeptide |
| 27 | <223> Synthetic polypeptide |
| 28 | <223> Synthetic polypeptide |
| 29 | <223> Synthetic polypeptide |
| 30 | <223> Synthetic polypeptide |

### Sequence Listing Free Text

The following information is provided for sequences containing free text under numeric identifier <223>.

1. A method of treating or reducing the progression of osteoarthritis (OA) comprising administering to a mammalian subject having OA an effective amount of a composition comprising an antibody or binding fragment thereof that binds leucine-rich repeat-containing protein 15 (LRRC15) in an amount sufficient to inhibit or suppress the activity of LRRC15.

2. The method according to claim 1, wherein said composition further comprises a pharmaceutically acceptable excipient or carrier.

3. The method according to claim 1, wherein said antibody or fragment comprises a heavy chain variable sequence of SEQ ID NO: 9, 11, 13, 15, 17, 19, or 21.

4. The method according to claim 3, wherein said antibody or fragment comprises a light chain variable sequence of SEQ ID NO: 10, 12, 14, 16, 18, 20, or 22.

5. The method according to claim 1, wherein said antibody or fragment comprises a heavy chain amino acid sequence of SEQ ID NOS: 23, 24, or 25.

6. The method according to claim 1, wherein said antibody or fragment comprises a heavy chain amino acid sequence of SEQ ID NOS: 30, 26, 27, 28, or 30.

7. The method according to one of claim 5 or 6, wherein said antibody or fragment comprises a light chain of SEQ ID NO:8 or 29.

8. The method according to claim 1, wherein the antibody or fragment comprises three heavy chain CDRs from the heavy chain full length or variable sequences of SEQ ID NO: 9, 11, 13, 15, 17, 19, 21, 23, 24, 25, 26, 27, 28 or 30.

9. The method according to claim 8, wherein the antibody or fragment comprises three light chain CDRs from the light chain full length or variable sequences of SEQ ID NO: 8, 10, 12, 14, 16, 18, 20, or 22, or 29.

10. The method according to any one of claim 3 or 4, wherein said antibody or fragment comprises a human heavy chain or light chain framework region of isotype IgG, IgG1, or IgM or IgY.

11. The method according to any one of claim 3 or 4, wherein the fragment is a single chain or single chain Fv fragment.

12. The method according to any one of claims 1 to 11 wherein said composition comprises one or more different said antibodies or fragments thereof.

13. The method according to any one of claims 1 to 12, wherein the composition is administered in vivo as a single dose.

14. The method according to claim 13, wherein the composition is administered as one or more booster doses.

15. The method according to claim 1, wherein the composition is administered by injection directly into a joint affected by OA.

16. The method according to claim 1, wherein the composition is administered systemically by oral, intramuscular, intraperitoneal, intravenous, intra-nasal administration, sublingual administration or intranodal administration or by infusion.

17. The method according to claim 1, wherein the subject is a human.

18. The method according to claim 2, wherein the carrier comprises a nanocarrier or nanoparticle suitable for direct injection into a joint.

19. The method according to any one of claims 1-18, where the composition is administered at a dose ranging from about 0.01 mg/kg to about 6 mg/kg.

20. A method of treating an arthritic joint comprising injecting into the joint of a mammalian subject having symptoms of fibrosis or osteoarthritis an effective amount of a composition comprising an antibody or binding fragment thereof that binds leucine-rich repeat-containing protein 15 (LRRC15) in an amount sufficient to inhibit or suppresses the activity of LRRC15.

21. The method according to claim 20, wherein said subject is human.

22. The method according to claim 20, wherein said osteoarthritis is at an early stage.

23. A method of treating or reducing the progression of osteoarthritis (OA) comprising administering to a subject having OA an effective amount of a composition that blocks, antagonizes, or inhibits the expression, induction, activity, or methylation, of the LRRC15 gene.

24. The method according to claim 23, comprising administering to a human having OA an effective amount of at least one compound, construct or composition that inhibits the expression or activity of the LRRC15 gene or modifies or silences the expression of LRRC15 gene in vivo.

25. The method according to claim 23, wherein said composition is an RNA or DNA construct that inhibits the expression of the LRRC15 gene.

26. The method according to claim 25, wherein said construct comprises a nucleic acid molecule that inhibits the translation or transcription of the LRRC15 gene.

27. The method according to claim 25, wherein said construct is a recombinant virus or virus-like particle that expresses an LRRC15 antagonist, a DNA construct that expresses an LRRC15 antagonist, an siRNA, shRNA or a CRISPR construct designed to interrupt or modify expression, transcription, or activity of the LRRC15 gene in vivo.

28. The method according to any one of claims 23 to 27, wherein the composition is administered in a single dose or as one or more booster doses.

29. The method according to claim 23, wherein the composition is administered systemically by oral, intramuscular, intraperitoneal, intravenous, intra-nasal administration, sublingual administration or intranodal administration or by infusion.

30. The method according to claim 23, wherein the composition is administered by injection directly into a joint affected by OA.

31. The method according to claim 23, wherein the composition comprises a small molecule inhibitor that targets LRRC15 protein or LRRC15 gene directly, or a salt, enantiomer, or prodrug thereof.

32. The method according to claim 23, wherein said composition further comprises a pharmaceutically acceptable excipient or carrier.

33. The method according to any one of claims 23 to 32, further comprising administering to said subject a methylation modifying drug.

34. A method of treating an arthritic joint comprising injecting into the joint of a mammalian subject having osteoarthritis an effective amount of a composition that blocks, antagonizes, or inhibits the level or activity, of the LRRC15 protein in vivo.

35. The method according to claim 23, wherein said subject is human.

36. The method according to claim 23, wherein said osteoarthritis is at an early stage.

37. A method for diagnosis of early-stage osteoarthritis in a mammalian subject, the method comprising obtaining a sample of synovial fluid or joint tissue from a subject, contacting said sample with a diagnostic reagent having a detectable label that measures the level of LRRC15 protein in the sample of a subject; wherein an increase in the level of LRRC15 protein as compared to a control level indicates the presence of early stage or progressing osteoarthritis.

38. The method according to claim 37 further comprising blocking further progression of osteoarthritis by administering a therapeutic agent that binds or inhibits further activity of LRRC15 protein.

39. A composition comprising an antibody or binding fragment thereof that binds leucine-rich repeat-containing protein 15 (LRRC15) for administration in an effective amount to a mammalian subject having osteoarthritis (OA) for treating or reducing the progression of the OA.
